(19) United States
(12) Patent Application Publication
(10) Pub. No.: US 2009/0311795 A1
(43) Pub. Date: Dec. 17, 2009

(54) BILATERAL CONTROL OF FUNCTIONS TRADITIONALLY REGULATED BY ONLY SEROTONIN OR ONLY DOPAMINE

(76) Inventor: Martin C. Hinz, Duluth, MN (US)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 12/483,727

(22) Filed: Jun. 12, 2009

Related U.S. Application Data
(63) Continuation-in-part of application No. 12/400,291, filed on Mar. 9, 2009.
Application No. 12/400,291 is a continuation of application No. 11/759,732, filed on Jun. 7, 2007, now abandoned.
Application No. 12/400,291 is a continuation-in-part of application No. 12/058,338, filed on Mar. 28, 2008.
Application No. 12/058,338 is a continuation of application No. 10/394,597, filed on Mar. 21, 2003, now abandoned.
Application No. 10/394,597 is a continuation-in-part of application No. 12/058,338, filed on Mar. 28, 2008.
Application No. 12/058,338 is a continuation of application No. 10/394,597, filed on Mar. 21, 2003, now abandoned.
Application No. 10/394,597 is a continuation-in-part of application No. 11/282,965, filed on Nov. 18, 2005.
Application No. 11/282,965 is a continuation-in-part of application No. 10/785,158, filed on Feb. 23, 2004, now abandoned.
(60) Provisional application No. 61/061,257, filed on Jun. 13, 2008.
(60) Provisional application No. 60/811,844, filed on Jun. 8, 2006.
(60) Provisional application No. 60/366,933, filed on Mar. 22, 2002.
(60) Provisional application No. 60/366,983, filed on Mar. 21, 2002.
(60) Provisional application No. 60/449,229, filed on Feb. 21, 2003.
(60) Provisional application No. 60/715,644, filed on Sep. 9, 2005.

(51) Int. Cl. G01N 33/00

(52) U.S. Cl. 436/98; 436/96; 436/111

(57) ABSTRACT

Methods of using amino acid precursors of the serotonin and catecholamine neurotransmitter systems and laboratory urinary assay of serotonin and catecholamine neurotransmitter levels for optimal treatment of transporters for neurotransmitters, and or neurotransmitter dysfunction and dysfunction of systems regulated or controlled by the serotonin and/or catecholamine neurotransmitter systems. The methods may also include determining a urinary neurotransmitter phase response to a change in dosing of supplemental amino acid precursors of the serotonin and catecholamine neurotransmitters to optimally treat neurotransmitter transporters, neurotransmitter dysfunction and dysfunction of systems regulated or controlled by the serotonin and/or catecholamine neurotransmitter systems.

### CROSS-REFERENCE TO RELATED APPLICATIONS

This application claims priority to provisional application U.S. Patent Application No. 61/061257, entitled Optimizing Organic Cation Transporter Regulatory Functions filed Jun. 13, 2008 the entire contents of which is incorporated by reference herein in its entirety.

This application is a continuation-in-part application of U.S. patent application Ser. No. 12/400291, entitled “Administration of Dopa Precursors with Sources of Dopa to Effectuate Optimal Catecholamine Neurotransmitter Outcomes”, filed Mar. 9, 2009, which is a continuation of U.S. patent application Ser. No. 11/759732 filed Jun. 6, 2007 which claimed priority to provisional U.S. Patent Application No. 60/811844 filed Jun. 8, 2006. U.S. patent application Ser. No. 12/400291 is also a continuation-in-part of application Ser. No. 12/058338 filed Mar. 28, 2008 which is a continuation of U.S. patent application Ser. No. 10/394597 filed Mar. 21, 2003 which claimed priority to provisional U.S. Patent application No. 60/366933 filed Mar. 21, 2002, the entire contents all of which are incorporated herein in by reference their entireties.

The present application is also a continuation-in-part application of U.S. patent application Ser. No. 12/058338, entitled “Serotonin and Catecholamine System Segment Optimization Technology”, filed Mar. 28, 2008, which is a continuation of U.S. patent application Ser. No. 10/394597 filed Mar. 21, 2003 which claims the benefit of provisional U.S. Patent application No. 60/366983 filed Mar. 21, 2002.

The present application is also a continuation-in-part application of U.S. patent application Ser. No. 11/282965, entitled “Serotonin and Catecholamine Segment Optimization technology” filed Nov. 18, 2005 which is a continuation-in-part of the inventor's co-pending application provisional patent application Ser. No. 10/785,158, filed Feb. 23, 2004, entitled, “Serotonin and Catecholamine System Segment Optimization Technology” which claims priority to provisional U.S. Patent Application No. 60/449,229, filed on Feb. 21, 2003, and U.S. patent application Ser. No. 11/282965 claims priority to U.S. Provisional Application No. 60/715,644, filed on Sep. 9, 2005, the contents all of which are incorporated herein in their entirety by reference in their entities.

### 37 C.F.R. §1.71(e) AUTHORIZATION

A portion of the disclosure of this patent document contains material which is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### FIELD OF THE INVENTION

The subject of this invention relates to serotonin and/or dopamine regulatory functions, neurohormone functions, paracrine functions and autocrine functions. For the purpose of this application, the term “monoamine: refers to serotonin and/or dopamine.

Serotonin and dopamine are synthesized from amino acid precursors. The amino acid precursors of serotonin are L-tryptophan and L-5-Hydroxytryptophan (5-HTP). The amino acid precursors of dopamine are phenylalanine, N-acetyl-tyrosine, tyrosine, and L-dopa. Serotonin and dopamine do not cross the blood brain barrier. The amino acid precursors of serotonin and dopamine do cross the blood brain barrier.

The present invention also relates, generally, to biomedical technology. More particularly, the invention relates to a technology for optimizing the serotonin and catecholamine functions regulated by administration of amino acid precursors of the serotonin and catecholamine systems in conjunction with serial laboratory assays of dopamine and serotonin systems. The invention also relates to effective compositions generally recognized as safe methods, therapies and techniques for managing the transporters and phases of urinary serotonin and dopamine responses in subjects with serotonin and catecholamine systems, in order to optimize individual and group outcomes in the treatment of monoamine dysfunction. The compositions, methods and techniques of the invention have broad applicability with respect to monoamine dysfunction, including disease. The compositions, methods, and techniques may also be useful in other fields.

### BACKGROUND OF THE INVENTION

With administration of amino acid precursors of the serotonin and/or catecholamine system there is an increase in monoamine levels of those systems in the system as a whole. Prior to this work laboratory measurement of the dopamine and the serotonin system (hereafter referred to collectively as “The System”) in correlation with amino acid response for clinical applications had not been calibrated for use. Instead, utilization of amino acid precursors of the serotonin and catecholamine systems produced random, mixed, and inconsistent results. The present invention provides a methodology for performing meaningful serotonin and dopamine laboratory assays in support of amino acid therapy in the treatment of the serotonin and catecholamine systems. This invention is a preferred step in any process where manipulations of the serotonin and dopamine-catecholamine systems are of consideration.

The catecholamine and serotonin are synthesized in the peripheral and central nervous systems, the kidneys, the liver, the gastrointestinal tract, the lungs, and any other place where the enzymes responsible for synthesis (i.e., tyrosine hydroxylase, tryptophan hydroxylase, and L-aromatic amino acid decarboxylase responsible for the conversion of amino acid precursors to serotonin and dopamine) are located. Pathways for serotonin and catecholamine synthesis are as follows:

Serotonin Synthesis:

L-Tryptophan→L-5-Hydroxytryptophan→Serotonin

Catecholamine Synthesis:

L-Tyrosine→L-Dopa→Dopamine→Norepinephrine→Epinephrine

Monoamine dysfunction associated with the catecholamine and/or serotonin systems may include, but is not limited to, depression, anxiety, panic attacks, migraine headache, obesity, bulimia, anorexia, premenstrual syndrome, menopause, insomnia, hyperactivity, attention deficit disorder (ADD), impulsivity, obsessionality, inappropriate aggression, inappropriate anger, psychotic illness, obsessive compulsive disorder (OCD), fibromyalgia, addictions, sexual dysfunction, chronic fatigue syndrome, chronic pain states, adrenal fatigue/burnout, attention deficit hyperactivity disorder (ADHD), Parkinsonism, traumatic brain injury, claustrophobia, tension headaches, nocturnal myoclons irritable bowel syndrome/Chron's disease, states of decreased cognitive function such as dementia and Alzheimer's disease, states associated with aging such as deterioration of organ systems innervated by the serotonin and/or catecholamine systems and deterioration of cognitive functions, hormone dysfunction problems innervated by the serotonin and/or catecholamine systems; cortisol dysfunction related problems, and neurotransmitter reaction to chronic stress.

Applicant has disclosed therapies for treatment of obesity and for serotonin and catecholamine system segment optimization technology in the following U.S. patents and published U.S. patent applications: U.S. Pat. No. 6,660,777 issued Dec. 9, 2003; U.S. Pat. No. 6,548,551, issued Apr. 15, 2003; U.S. Pat. No. 6,403,657, issued Jun. 11, 2002; U.S. Pat. No. 6,384,088, issued May 7, 2002; U.S. Pat. No. 6,759,437, issued on Jul. 6, 2004; U.S. Patent Application Publication No. U.S. 2003/0181509 A1, published Sep. 25, 2003; U.S. Patent Application Publication No. U.S. 2002/0094969 A1, published Jul. 18, 2002; U.S. Patent Application Publication No. U.S. 2002/0072537 A1, published Jun. 13, 2002; U.S. Patent Application Publication No. U.S. 2002/0065311 A1, published May 30, 2002; U.S. Patent Application Publication No. U.S. 2002/0040054 A1, published Apr. 4, 2002; U.S. Patent Application Publication No. U.S. 2002/0025972 A1, published Feb. 28, 2002; U.S. Patent Application Publication No. U.S. 2004/0101575 A1, published May 27, 2004; and U.S. Patent Application Publication No. U.S. 2005/0233008 A1, published Oct. 20, 2005.

Beyond functioning as neurotransmitters serotonin and dopamine carry out other functions in the system. It has not been known that the functions regulated by serotonin or dopamine may be dependent with one another when dopamine regulates a specific list of functions and serotonin regulates a specific list of functions. Serotonin and dopamine exist in two distinct states in the system, “the endogenous state” found when no amino acid precursors are administered and “the competitive inhibition state” found when amino acid precursors are administered. The “competitive inhibition state” relates to synthesis, metabolism, and transport of serotonin and dopamine. “Despite considerable advances in the understanding of basic transport pathways and mechanisms involved in the tubular secretion of organic compounds, there is still relatively little information on the regulation of this transport” (Kidney International, Vol. 59 (2001), pp. 17-30). While “the competitive inhibition state” has been identified, so little is known that the literature notes, “The functional relevance of the competitive inhibitory effect of L-DOPA upon the decarboxylation of L-5-HTP by AAAD is most probably meaningless” (BrMsh Journal of Pharmacology (1996) 117, 1187 1192). The teaching of this invention involves a method of placing both serotonin and dopamine in the competitive inhibition state then utilizing this joint state to allow for manipulation of functions by only dopamine or only serotonin or by both serotonin and dopamine.

A need is believed to exist for the present invention. All U.S. patents and patent applications, and all other published documents mentioned anywhere in this application are hereby incorporated by reference in their entirety.

The nervous system is a human body's key communications network. Along with the endocrine system, it provides most of the control functions of the body. The main parts of the nervous system are the brain, the spinal cord (which together with the brain makes up the central nervous systems (CNS), and the peripheral nervous system. The nerves are comprised of groups of neurons. Neurons transmit impulses or signals. Each neuron comprises a cell body or soma, dendrites that receive chemical signals from other neurons and axons that convey the signals as electrical impulses. A synapse is the junction point from one neuron to another. A great deal of signal control occurs at the synapse. In most chemical synapses, a first (pre-) neuron secretes a neurotransmitter into the synapse and this in turn acts on receptor proteins in the membrane of the next (post-) neuron. The transmitters may to excite the neuron, inhibit it, or modify its sensitivity in some other way.

Presently, 183 substances have been identified which can function as a neurotransmitter (synaptic transmitter) in the central nervous system. Master regulation of the neurotransmitters of the central nervous system neurotransmitters is attributed to the serotonin system and the catecholamine system.

Catecholamines include dopamine, norepinephrine and epinephrine. Both serotonin and catecholamines include relatively small molecules, which act relatively fast. These transmitters cause most of the acute responses of the nervous system, such as transmission of sensory signals to and inside the brain and motor signals back to the muscles. The catecholamines and serotonin are synthesized in the cytosol of presynaptic terminals or in other cells possessing the required enzymes such as the proximal convoluted renal tubule cells. Presynaptic terminals are small knobs, which lie primarily on the surface of the dendrites. The synthesized transmitters are absorbed by transmitter vesicles in the terminals. Transmitters are released from the terminals by an action potential mechanism and cross a small synaptic cleft where they act on the post synaptic membrane receptors are discussed above. After a transmitter is released at a nerve ending, it is either destroyed or removed by transport to prevent continued action. Removable mechanisms include diffusion of the transmitter out of the cleft, enzymatic destruction of the transmitter within the cleft itself, and transmitter re-uptake, which is the active transport back into the presynaptic terminal itself for reuse.

The catecholamine norepinephrine is secreted by many neurons whose cell bodies are located in the brain system and hypothalamus. It is believed to help control overall activity and mood of the mind. Norepinephrine is also secreted by most of the post ganglionic mehnrons of the sympathetic (visceral functions of the body such as arterial blood pressure, gastrointestinal activity, urination, swearing and body temperature) nervous systems, where it excites some organs and inhibits others. The catecholamine dopamine in the central nervous system is secreted by neurons that originate in the substantia nigra. The effect of dopamine is usually inhibition. Serotonin in the central nervous system is secreted by nuclei that originate in the median raphe of the brain stem. Serotonin acts as an inhibitor of pain pathways in the cord, and it is also believed to help control the mood and to regulate sleep through its role as a precursor of melatonin.

It is known from applicant's work that the serotonin system and the catecholamine system effectively work as one unit (hereinafter defined as The System). It is known from such work, that levels that are not high enough to effect needed electrical transport are associated with numerous diseases and illnesses. Dysfunction (which includes disease or illness, neurotransmitter dysfunction, sub-optimal performance of systems dependent on monoamines for regulation and function, or other malady relating to the catecholamine and/or serotonin systems) results from suboptimal transfer of electrical energy between the input of the pre-synaptic neuron and output of post-synaptic neurons and/or neuron bundles of The System.

Dysfunction of the neurons of the central nervous system, in general, give rise to diseases and symptoms related to psychiatric illness and master control centers such as eating disorders, Parkinsonism and the like. Dysfunction of neurons of the peripheral nervous system in general, produces end organ disease, sub-optimal results and dysfunction. The primary mechanism of dysfunction is a discrepancy between the electrical input to the neurons or neuron bundles and the output of the neurons or neuron bundles of The System. Anything that affects the electrical outflow of the neuron bundles to give a disproportion between the inflow and the outflow of electric energy can cause dysfunction. Examples of mechanisms and consideration of dysfunction include but are not limited to:

Nutritional deficiency

Increased metabolism secondary to drugs and substances

Hyperexcretion

Receptor regulation

System damage

Neurotransmitter levels lower than the threshold induce dysfunction.

Drugs that work with neurotransmitters do not work if there is insufficient neurotransmitters to work with. Drugs work with neurotransmitters of one system may not produce the desired effects if the neurotransmitter levels of the other system are too low. This supports the assertion that both systems must be functioning properly for The System to function normally and proper response to stimuli to be observed.

It has been observed in clinical situations where subjects have extremely low levels of neurotransmitters in one system with higher than normal urinary neurotransmitter levels in the other system, that no response is seen from drugs that exert their effects on the higher system, or that increased dosing of the drug was needed to effectuate the desired clinical response. Under these same circumstances sub-optimal response to the drug may be seen.

Only to a certain point can markedly increased levels of neurotransmitters in one system compensate for low levels of neurotransmitters in the other system and if the neurotransmitter levels in the other system are too low, no response from the increased system will be seen no matter how high the levels of neurotransmitters are in that system.

Hormone dysfunction is not optimal until the neurotransmitters controlling the system are optimized. Dysfunction of the serotonin and/or dopamine of The System contributes greatly to hormone problems. Optimal results can be obtained in addressing systems innervated by the System. First, the neurotransmitters of the system are optimized. For example, it was found that if hormone replacement therapy was administered to subjects prior to optimization of The System and The System was then optimized, on re-evaluation of the hormone system, it was found that hormone replacement in general was affected to the point where it had to be once again addressed and in some cases was no longer needed. Optimizing the serotonin and dopamine of The System prior to correcting problems of with systems innervated by The System is preferred.

The benefits of balancing and optimizing neurotransmitter levels and neurotransmitter transporters include:
1. Reestablishing a clinic response from a drug, substance, or compound that is dependent on neurotransmitters for their effects and has developed a tachyphylaxis and quit working.
2. Establishing a clinical response in circumstances where no clinical response is seen in subjects from initiation of the drug, substance or compound that is dependent on neurotransmitters for their effects.
3. Optimizing or enhancing the response from drugs, substances, and compounds that is dependent on neurotransmitters for their effects and work with the catecholamine system and/or serotonin system.
4. Optimizing neurotransmitter levels for treatment and relief of symptoms relating to dysfunction of The System.
5. Inducing a display of new properties not previously known or seen in the past as neurotransmitters of the System interact with The System in states of dysfunction or states altered by forces outside The System to include but not limited to alterations by drugs, substances, compounds, or organisms introducing them into The System.
6. Establishing a side effect profile in use that is much lower than seen with use of the individual amino acid components or amino acid components no in proper balance.
Optimizing intervation regulation and function of other systems interacting with the system.

### SUMMARY OF THE INVENTION

The present disclosure describes methods of using amino acid precursors of the serotonin and catecholamine neurotransmitter systems and laboratory urinary assay for optimal treatment of monoamine dysfunction and dysfunction of systems regulated or controlled by the serotonin and/or catecholamine neurotransmitter systems.

In some embodiments, the invention pertains to a method of managing distinct urinary serotonin and dopamine responses in subjects with a serotonin and a catecholamine system, the method comprising performing a first laboratory assay on a subject that has consumed a first amino acid dosing of serotonin and catecholamine precursors, performing a second laboratory assay on a subject that has consumed a different second amino acid dosing of serotonin and catecholamine precursors, reviewing the first laboratory assay results, reviewing the second laboratory assay results, and adjusting the amino acid dosing range upon reviewing the first and second laboratory assay results to establish the associated serotonin and catecholamine in an effective therapeutic range.

In some embodiments, there is a need for only one laboratory assay to determine with certainty the next step approach needed for optimization of serotonin and dopamine. For example, synthesis of dopamine from tyrosine is regulated by the norepinephrine tyrosine hydroxylase feed back loop. Administration of tyrosine will move urinary dopamine from phase 1 to phase 2 then into phase 3 but no higher than 475 micrograms of dopamine per gram of creatinine in phase 3. This coupled with the fact that urinary serotonin and dopamine have never been observed in phase 1 simultaneously leads to the following ability to determine the phase of both urinary serotonin and dopamine with one assay. In a subject taking tyrosine and 5-HTP a urinary serotonin of 900 micrograms of serotonin per gram of creatinine with a urinary dopamine of 650 micrograms of dopamine per gram of creatinine is found. Since tyrosine will not establish levels of dopamine greater than 475 micrograms of dopamine per gram of creatinine in phase 3 the dopamine is phase 1. Since dopamine and serotonin are never in phase 1 at the same time the serotonin is phase 3. This is an example of how serotonin and dopamine phases are determined without the need for two assays while taking two different precursor dosings.

In some embodiments, the method can further comprise adjusting only the serotonin precursor. In some embodiments, the method can further comprise adjusting only the catecholamine precursor. In some embodiments, the method can further comprise adjusting both the serotonin and catecholamine precursors.

In some embodiments, the invention pertains to a method for managing distinct urinary serotonin and dopamine responses in subjects with a serotonin and catecholamine system, the method comprising performing a first urinary laboratory assay on a subject that has consumed a first amino acid dosing of serotonin and catecholamine precursors, performing a second urinary laboratory assay on the subject that has consumed a second amino acid dosing of serotonin and catecholamine precursors, reviewing the first urinary laboratory assay results, reviewing the second urinary laboratory assay results, and adjusting the amino acid dosing range to monitor the exertion of the serotonin and catecholamine into the urine of the subject.

In some embodiments, the method of adjusting an increase in both the serotonin and catecholamine precursors in a subject occurs when the urinary serotonin and catecholamine levels of serotonin or catecholamine decrease as the dosing of serotonin and catecholamine precursors are increased.

In some embodiments, the method of adjusting either an increase or decrease in both the serotonin and catecholamine precursors in a subject to stabilize the serotonin and catecholamine in the therapeutic range occurs when the urinary neurotransmitter levels of serotonin and catecholamine increase as the dosing of serotonin and catecholamine precursors are increased.

In some embodiments, the method of adjusting an increase in catecholamine precursors and a decrease in serotonin precursors in a subject occurs when the urinary serotonin increases and catecholamine decreases as the dosing of serotonin and catecholamine precursors are increased.

In some embodiments, the method of adjusting an increase in serotonin precursors and decrease in catecholamine precursors in a subject occurs when the urinary serotonin decreases and catecholamine increases as the dosing of serotonin and catecholamine precursors are increased.

In some embodiments, the method of adjusting an increase in serotonin precursors and decrease in catecholamine precursors in a subject occurs when the urinary serotonin remain relatively unchanged and catecholamine increases as the dosing of serotonin and catecholamine precursors are increased.

In some embodiments, the method of adjusting a decrease in serotonin precursors and increase in catecholamine precursors in a subject occurs when the urinary serotonin increase and and catecholamine remains relatively unchanged as the dosing of serotonin and catecholamine precursors are increased.

In some embodiments, the method of adjusting an increase in the serotonin precursors and a decrease in the catecholamine precursors in a subject occurs when the urinary serotonin and catecholamine increase as the dosing of serotonin and catecholamine precursors are increased but the serotonin precursors are in a therapeutic range and the catecholamine precursors are above the therapeutic range.

In some embodiments, the method of adjusting an increase in the catecholamine precursors and a decrease in the serotonin precursors in a subject occurs when the urinary serotonin and catecholamine increase as the dosing of serotonin and catecholamine precursors are increased but the catecholamine precursors are in a therapeutic range and the serotonin precursors are above the therapeutic range.

In some embodiments, the urinary serotonin and dopamine levels of a subject can each be established in any one of three phases of urinary response to amino acid precursor dosing.

In some embodiments, the urinary serotonin and dopamine levels of a subject can each be established in the same phase of urinary response to amino acid precursor dosing.

In some embodiments, the urinary serotonin and dopamine levels of a subject can each be established in separate phases of urinary response to amino acid precursor dosing.

In some embodiments, amino acid precursor dosing may be adjusted to manipulate each of the urinary serotonin and dopamine levels of a subject anywhere above, below, or in a therapeutic range in one of any three phases of urinary response to amino acid precursor dosing.

In some embodiments, a serotonin and dopamine assay in support of amino acid therapy is provided which insures that proper levels of monoamines are established and when used properly minimizes the risks of monoamine overload during use.

In some embodiments, a method of establishing at least one serotonin or dopamine status point in a subject comprising the steps of determining a subject's health status with respect to neurotransmitter and/or regulatory dysfunction, performing an assay of a body fluid of the subject to determine a serotonin and dopamine level in the fluid, and defining the assayed serotonin and dopamine levels in the fluid as at least one serotonin and/or dopamine status point is provided.

In some embodiments, a method of treating a subject for neurotransmitter or regulatory dysfunction, comprising the steps of performing a first assay of a body fluid of a subject to determine a serotonin or dopamine level in the body fluid, administering an amino acid precursor of a neurotransmitter to the subject, administering a second assay of a body fluid of the subject to determine whether the serotonin and dopamine level in the body fluid is within a predetermined therapeutic range of serotonin and dopamine levels is provided.

### BRIEF DESCRIPTION OF THE DRAWINGS

FIG. 1 summarizes Monoamine Neurotransmitters.

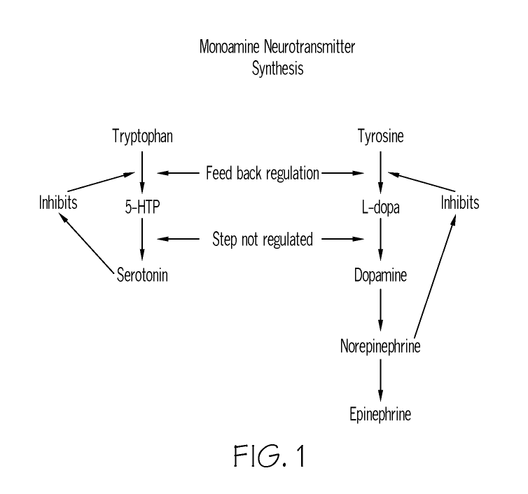

FIG. 2 summarizes Monoamine Neurotransmitter Functions.

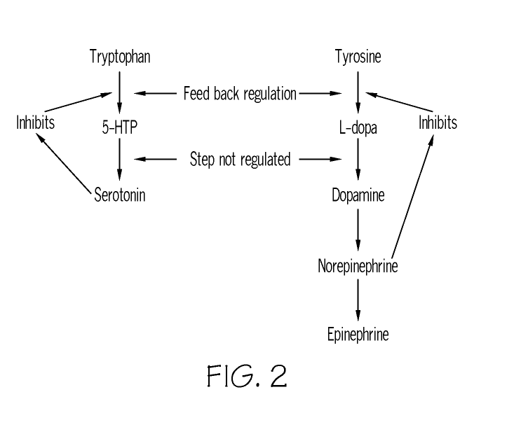

FIG. 3 summarizes Monoamine Neurotransmitter Synthesis.

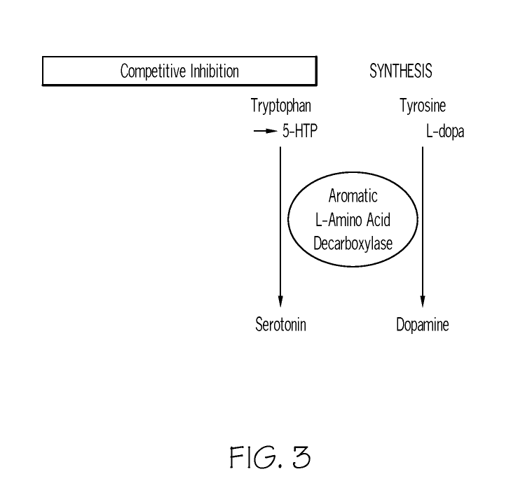

FIG. 4 summarizes Monoamine Neurotransmitter interaction with the Brain Barrier.

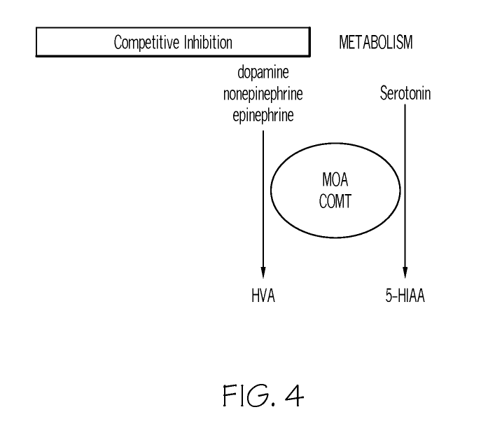

FIG. 5 summarizes Monoamine Neurotransmitter Uptake.

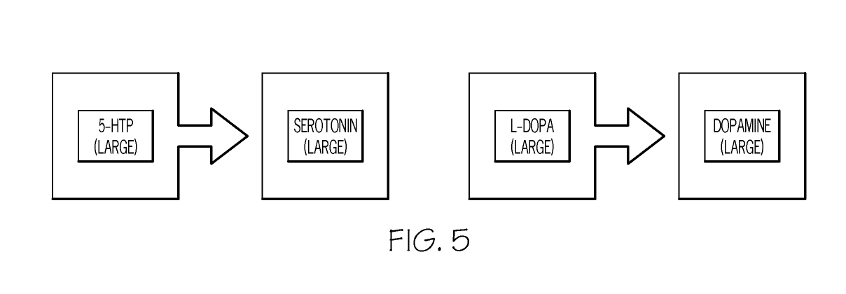

FIG. 6 summarizes Monoamine Neurotransmitter in the Competitive Inhibition State.

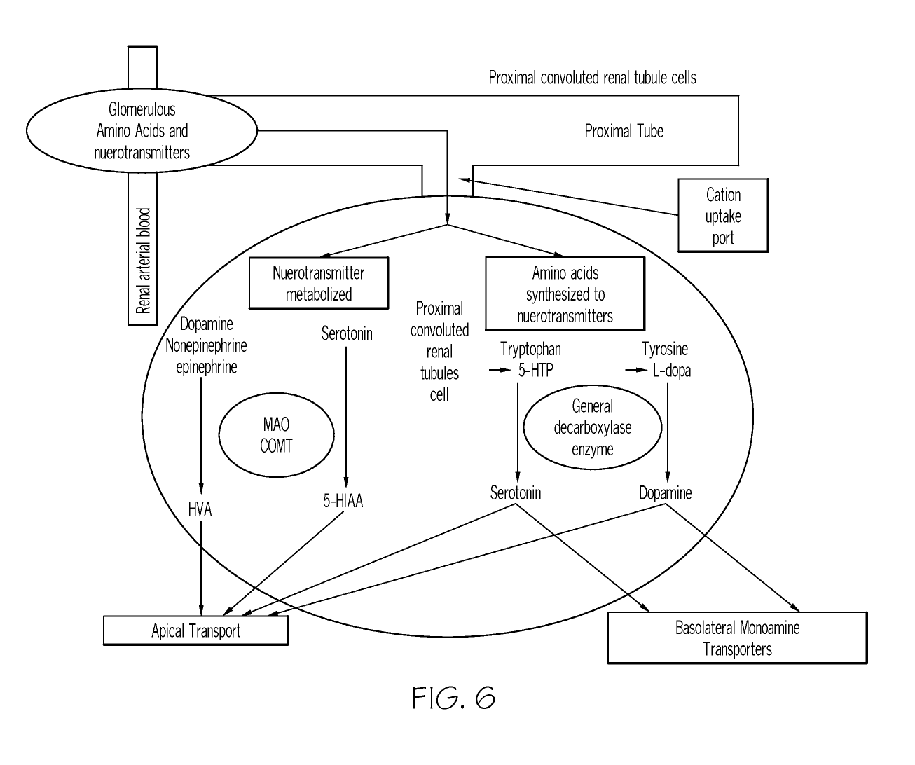

FIG. 7 summarizes Competitive Inhibition Synthesis.

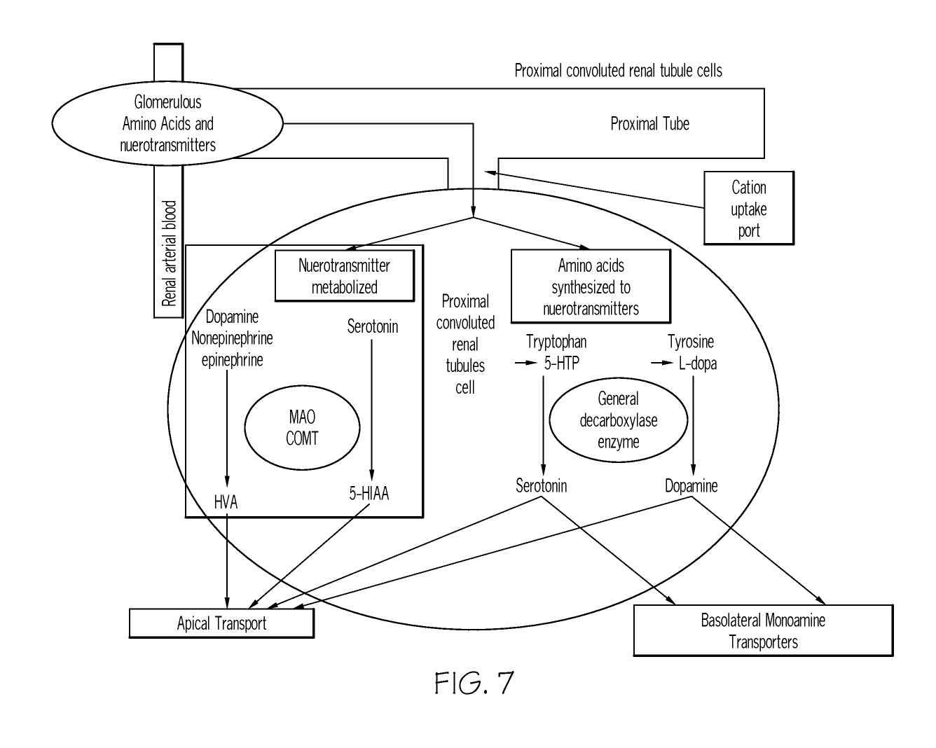

FIG. 8 summarizes Competitive Inhibition Metabolism.

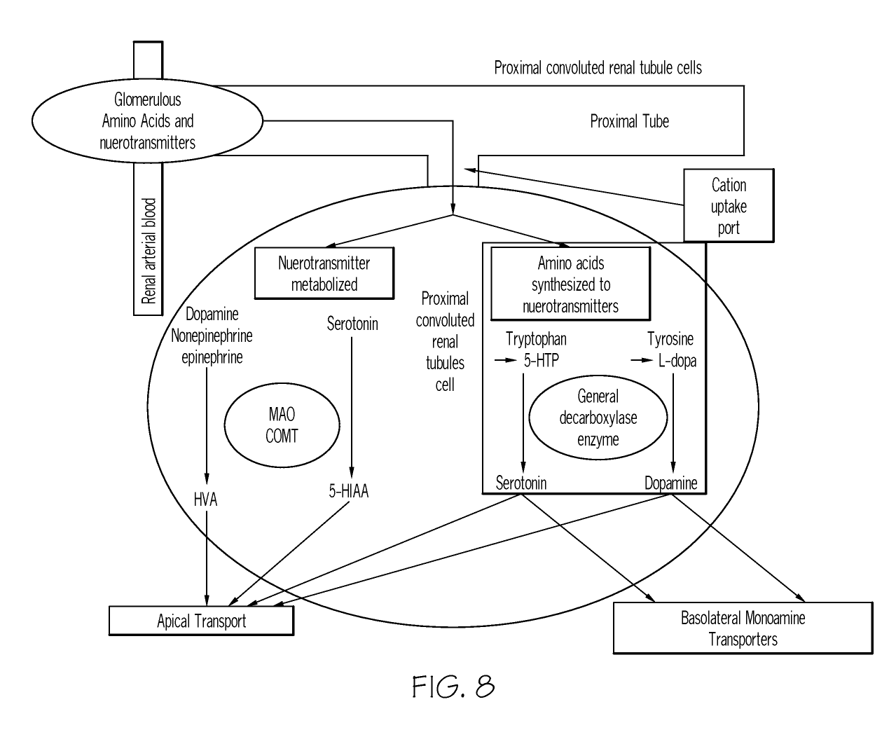

FIG. 9 summarizes Competitive Inhibition Uptake.

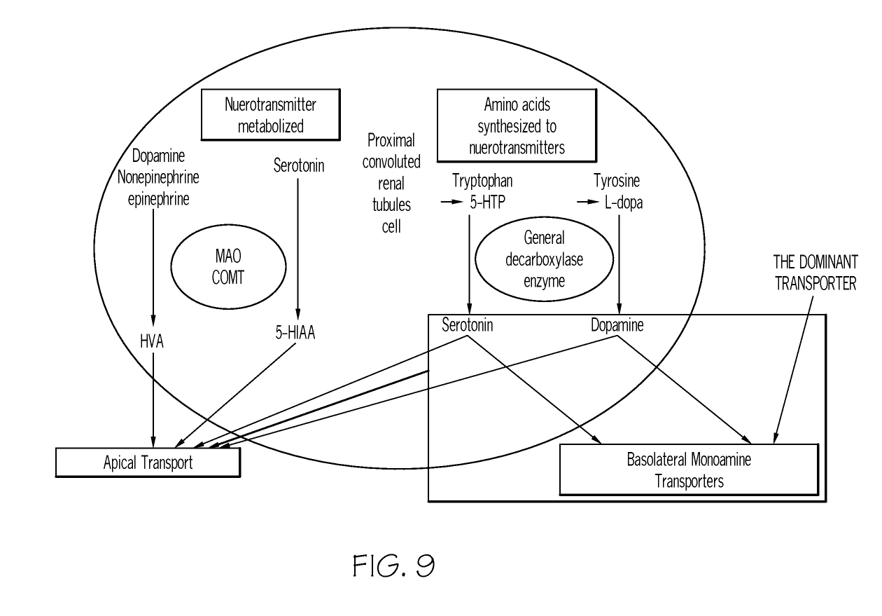

FIG. 10 is another summary of Competitive Inhibition Uptake.

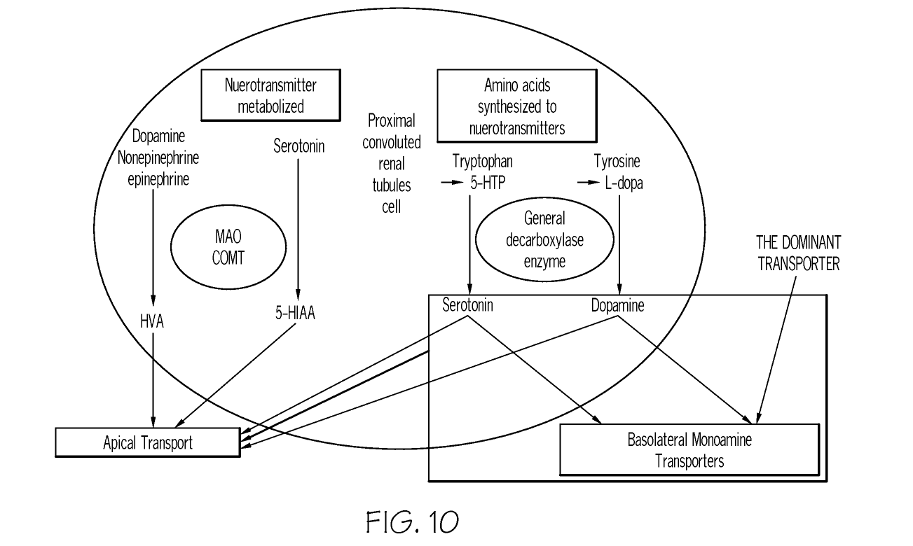

FIG. 11 is a summary for the balance of 5-HTP and L-Dopa.

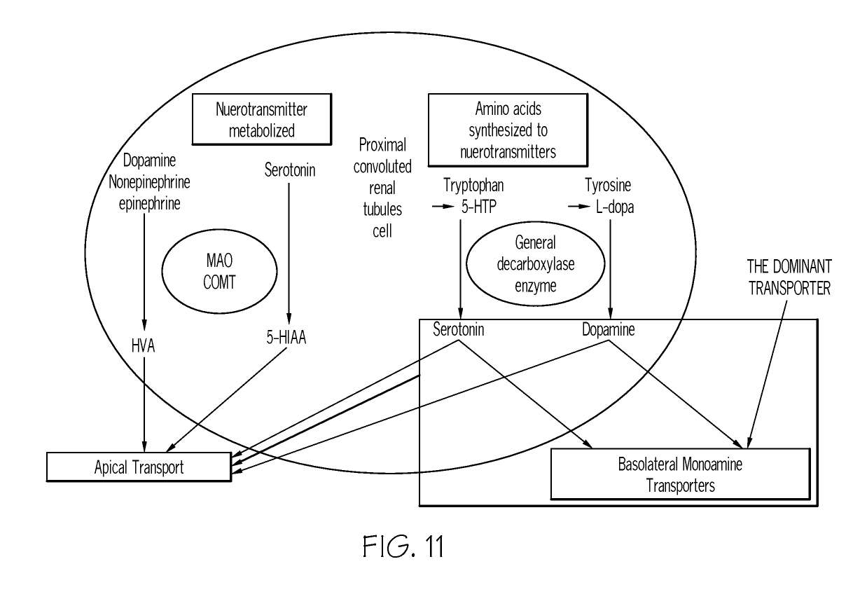

FIG. 12 summarizes Urinary Monoamine Neurotransmitters.

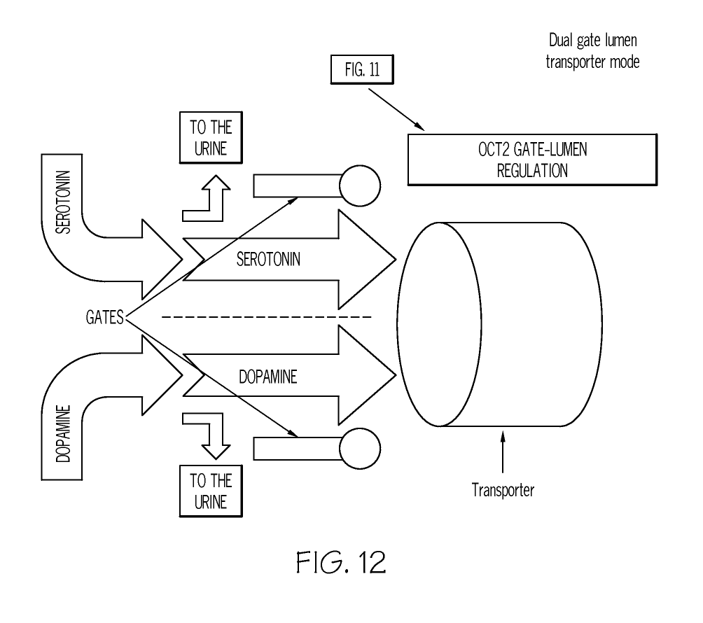

FIG. 13 summarizes Proximal Convoluted Renal Tubule Cells.

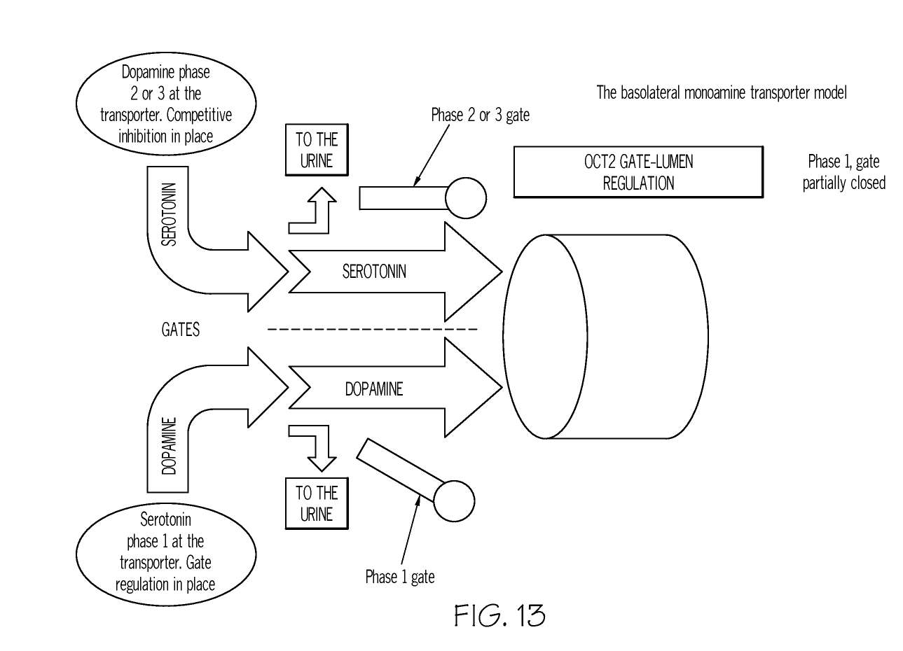

FIG. 14 is another summary of Proximal Convoluted Renal Tubule Cells.

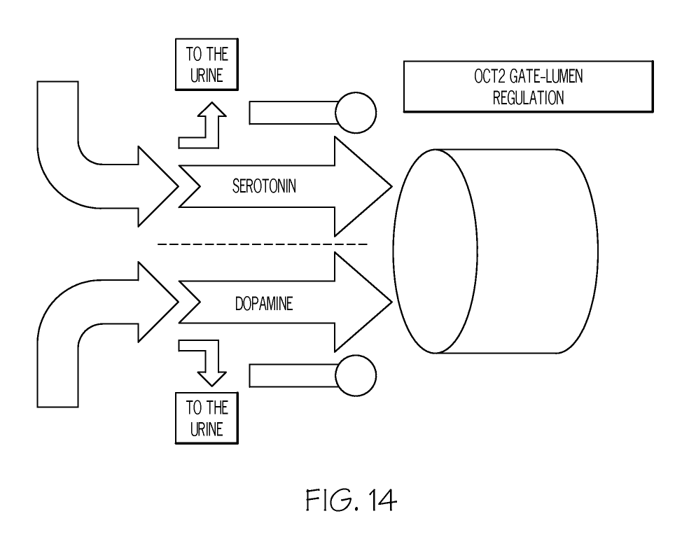

FIG. 15 is another summary of Proximal Convoluted Renal Tubule Cells.

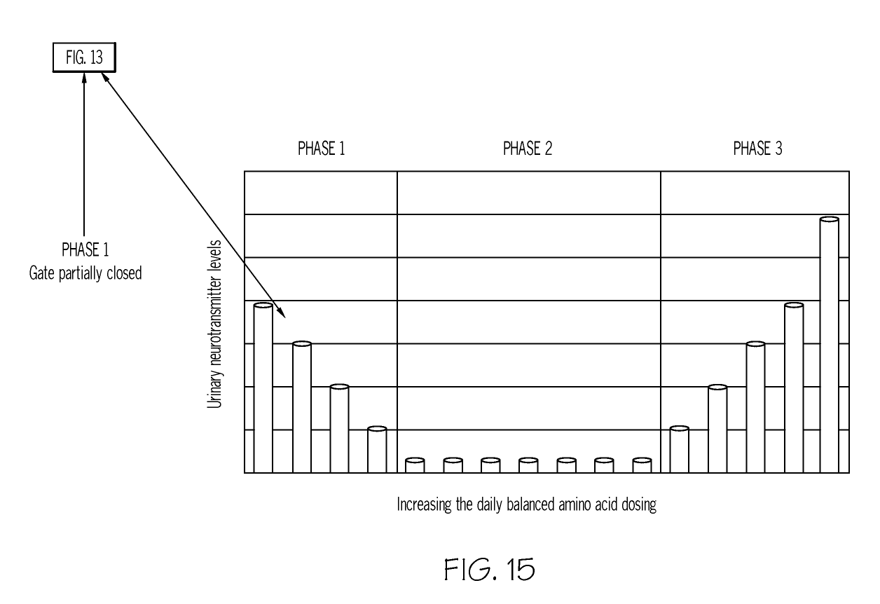

FIG. 16 is a summary of the Basolateral Monoamine Transporter and Apical Transporter.

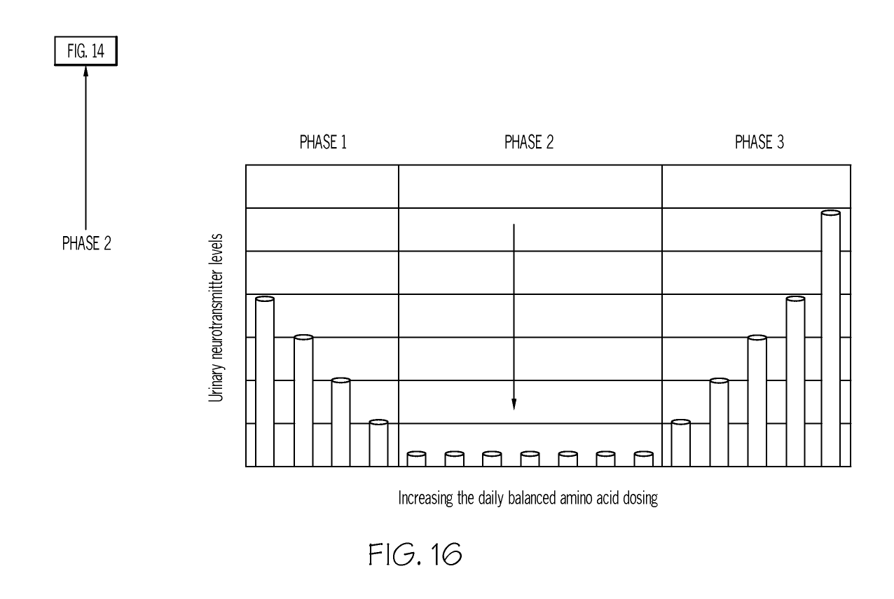

FIG. 17 is a summary of Urinary Assays in the Endogenous State.

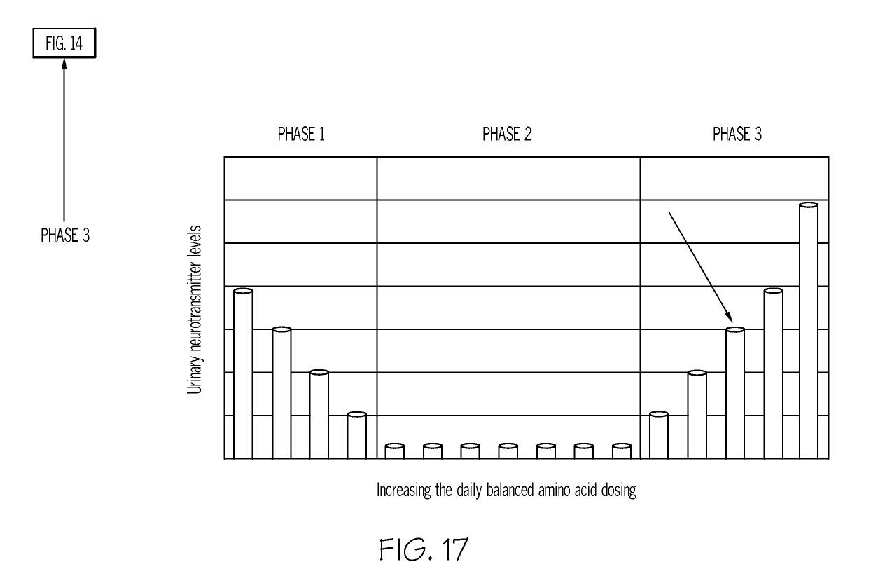

FIG. 18 is a summary of Assays in the Competitive Inhibition State.

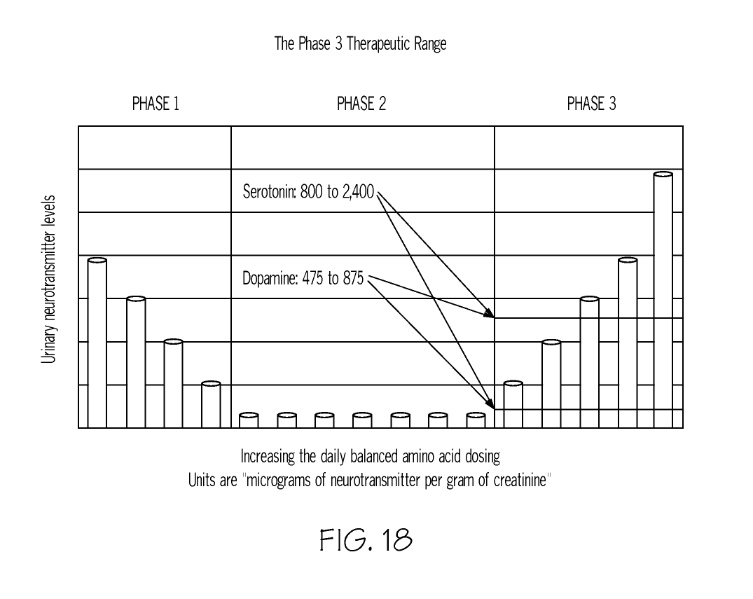

FIG. 19 is a summary of the Dual Gate Lumen Transporter Model.

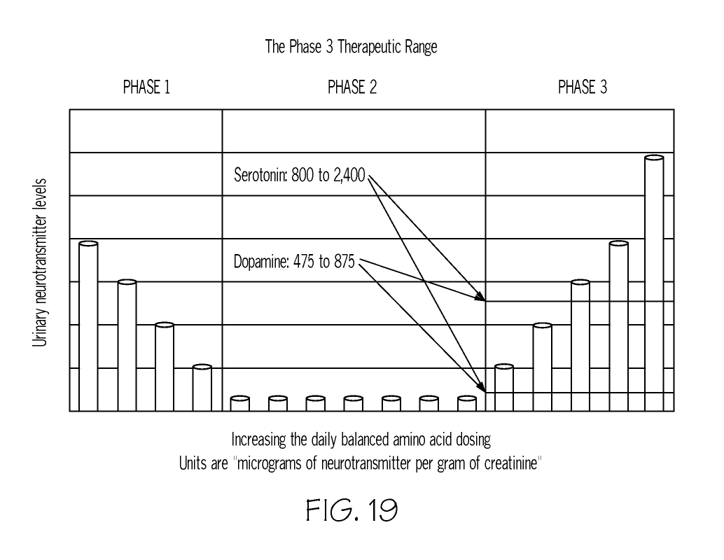

FIG. 20 is a summary of the Basolateral Monoamine Transporter Model.

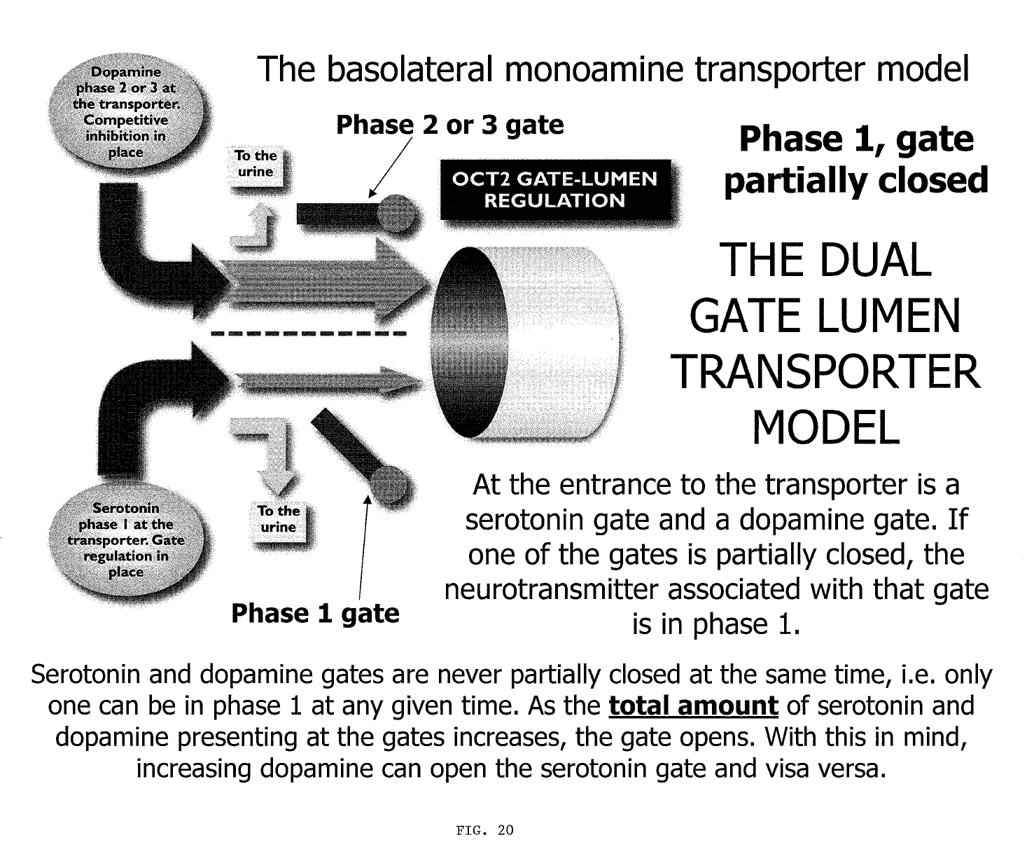

FIG. 21 is another summary of the Basolateral Monoamine Transporter Model.

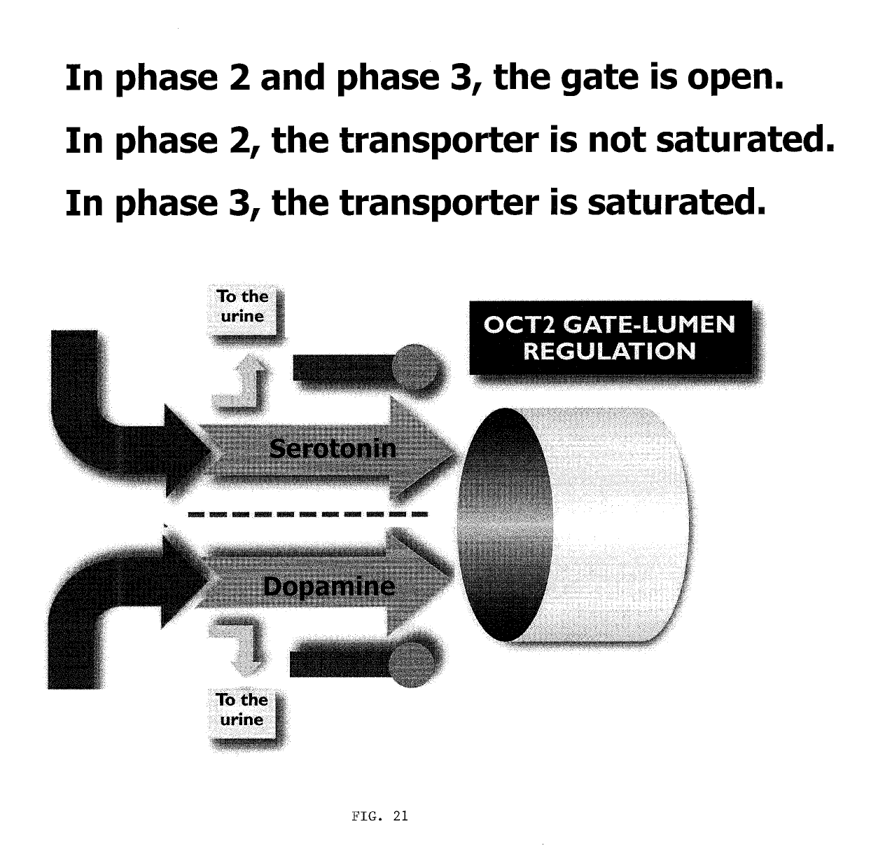

FIG. 22 is a summary of Laboratory Interpretation.

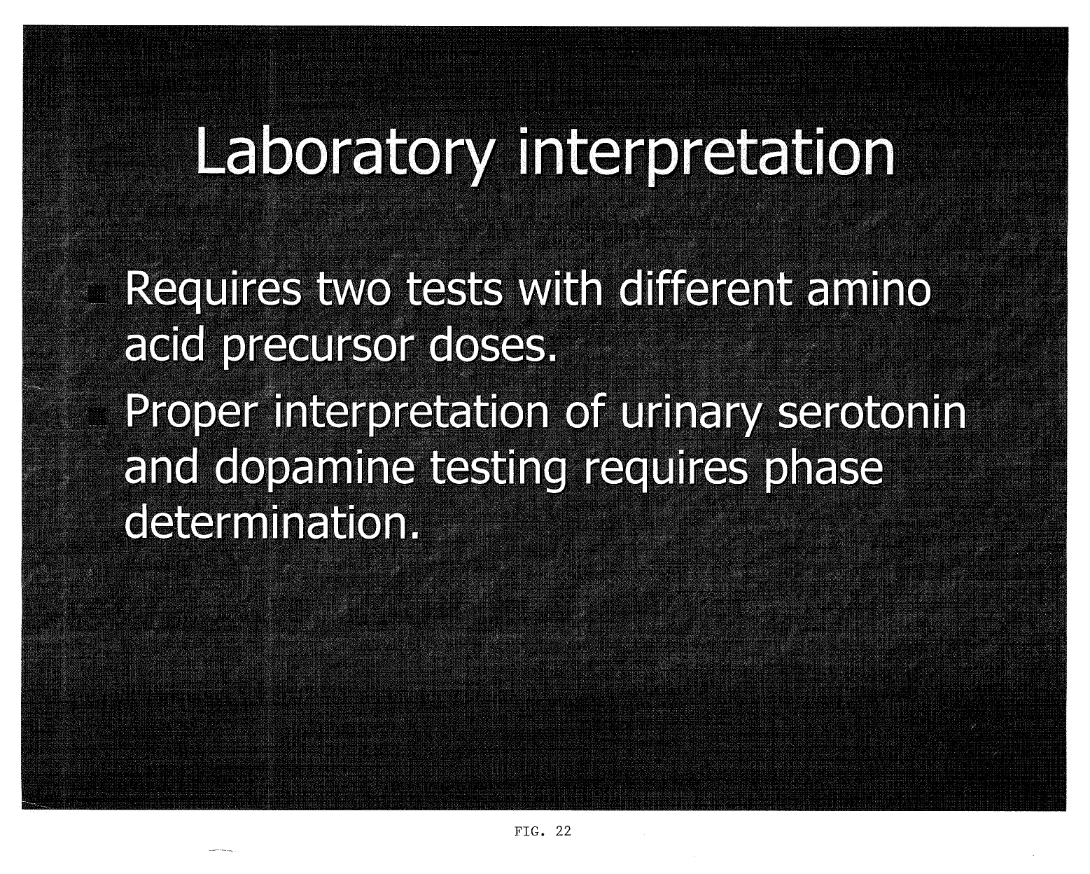

FIG. 23 is a summary of Amino Acid Dosing in Phase 1.

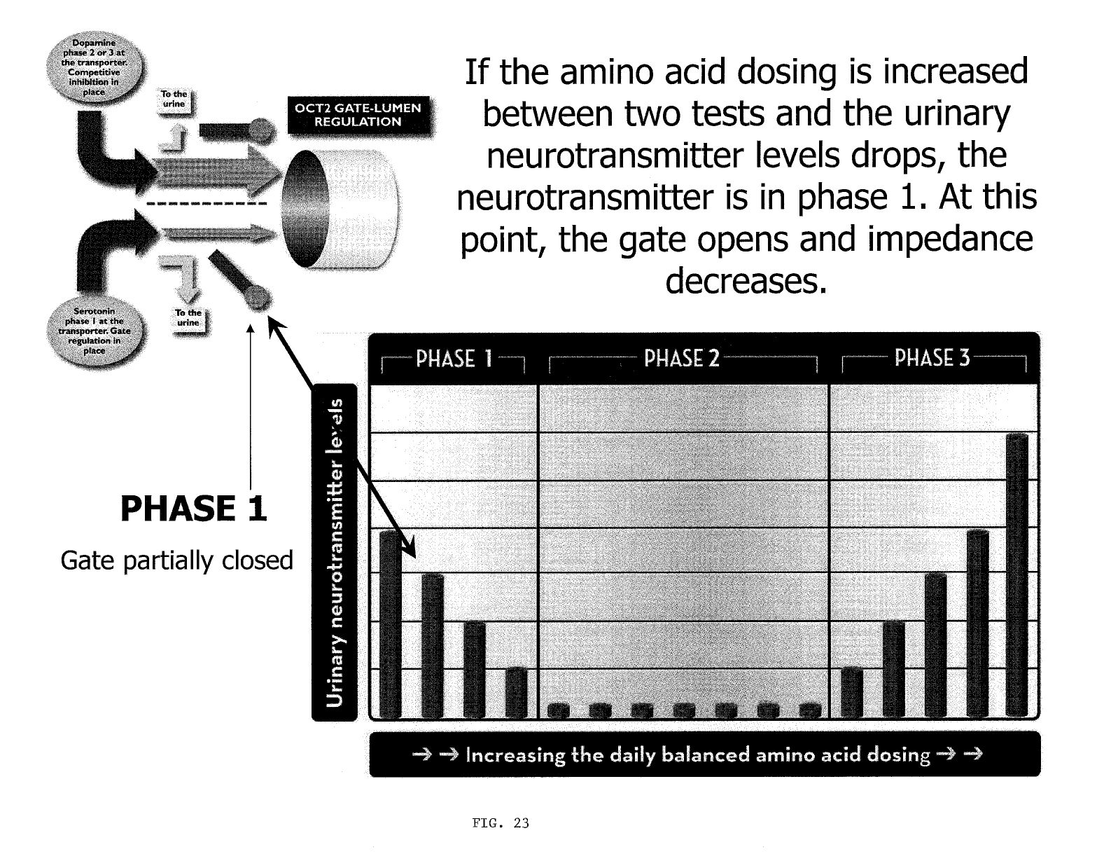

FIG. 24 is a summary of Amino Acid Dosing in Phase 2.

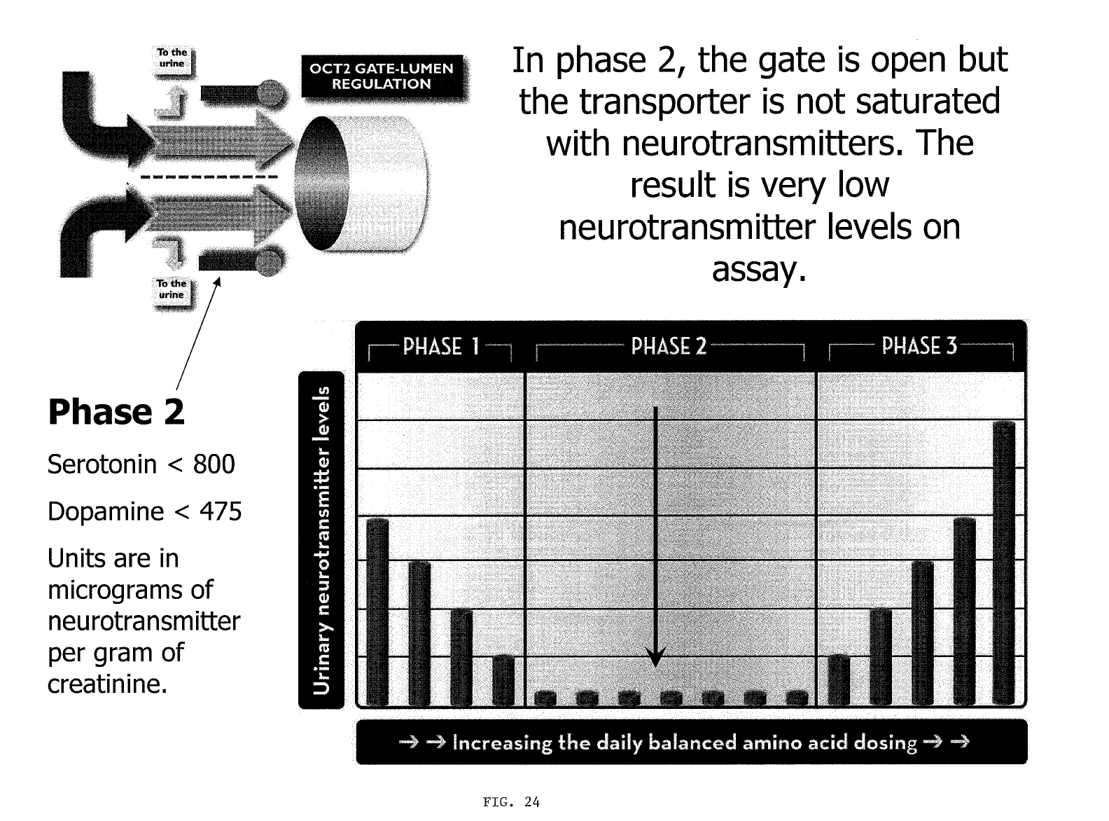

FIG. 25 is a summary of Amino Acid Dosing in Phase 3.

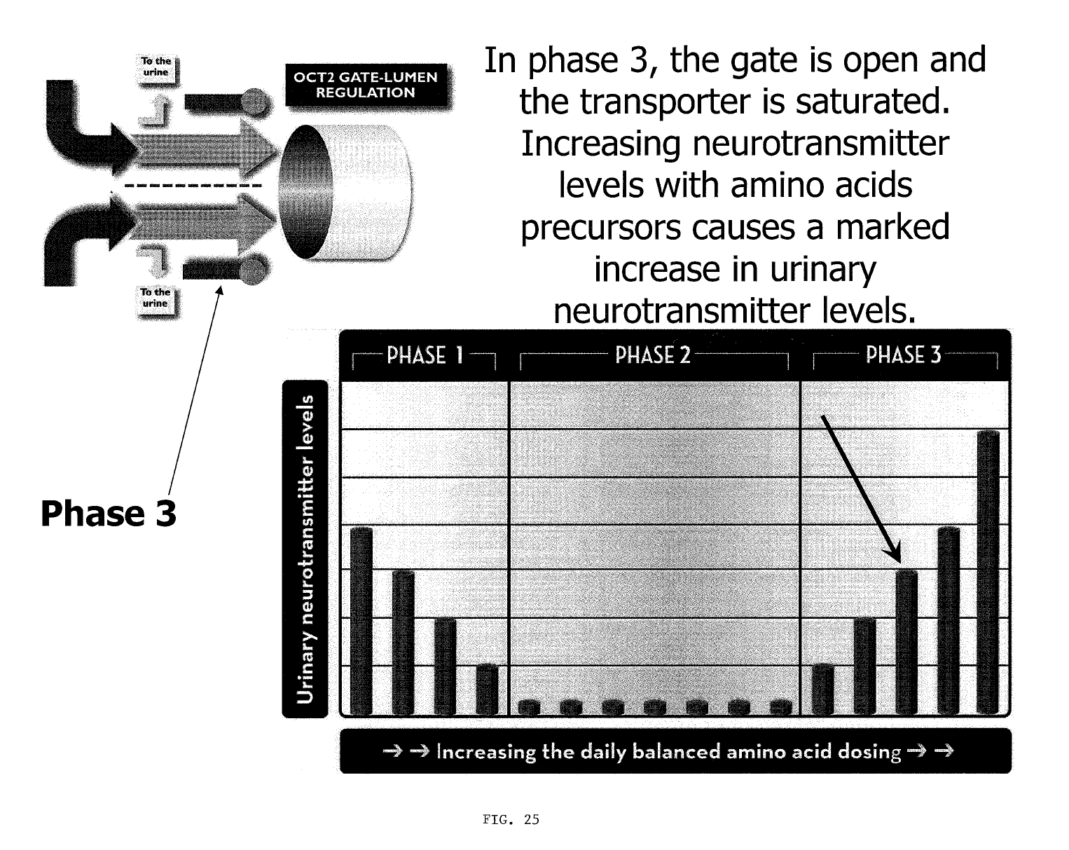

FIG. 26 is a summary of the Phase 3 Therapeutic Range.

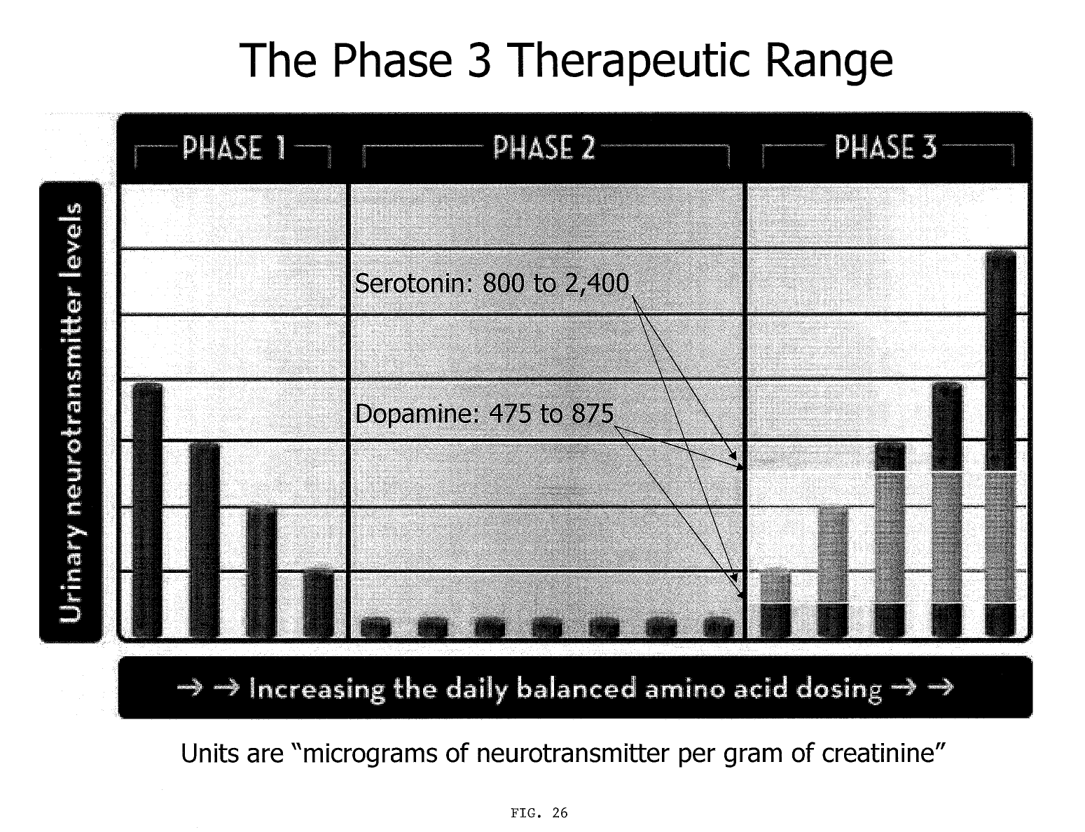

FIG. 27 is another summary of the Phase 3 Therapeutic Range.

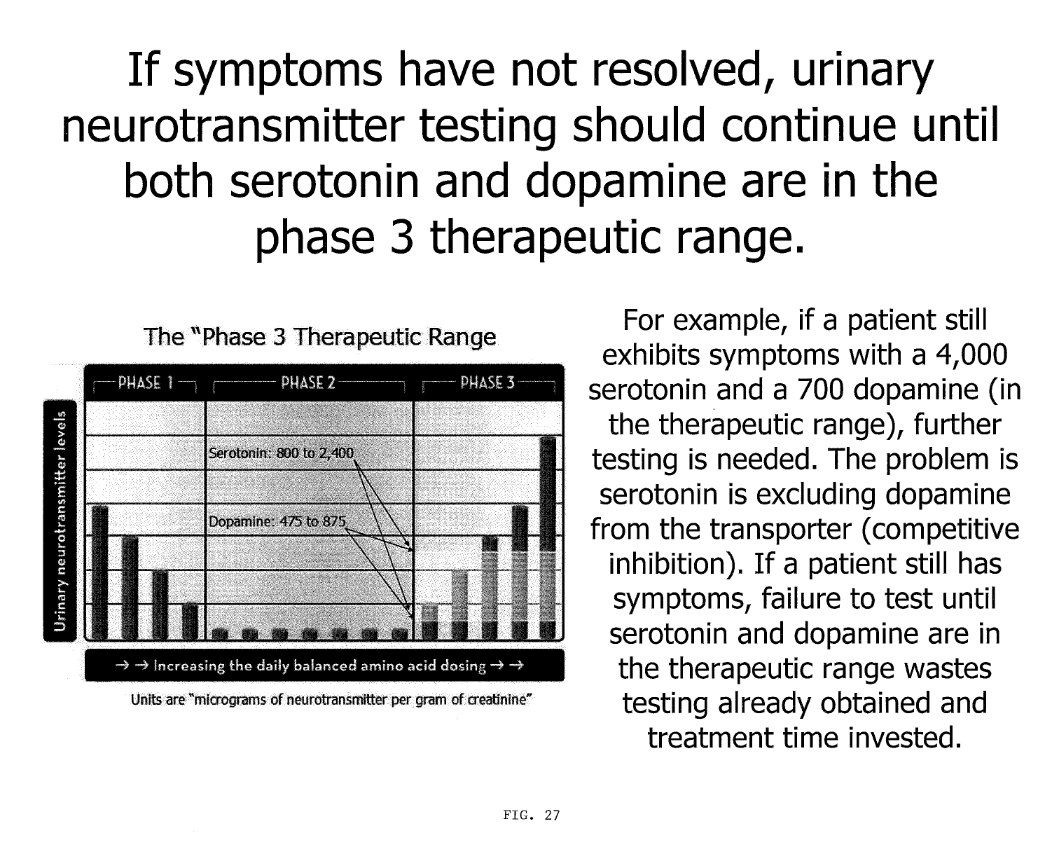

### DETAILED DESCRIPTION OF SOME OF THE DISCLOSED EMBODIMENTS

The embodiments of the invention described are intended to be illustrative and not to be exhaustive or limit the invention to the exact forms disclosed. The embodiments are chosen and described so that a person of ordinary skill in the art will be able to understand the invention and the manner and method of using it.

The teachings of this invention, in general, relate to optimizing individual and group outcomes in the treatment of serotonin and catecholamine (dopamine, norepinephrine, and epinephrine) systems and transporters of serotonin and dopamine in the management of dysfunction in human beings as guided by laboratory assay of monoamines. However, the teaching is useful in any life form where the catecholamine system and the serotonin system with renal systems are found, such as other animals (i.e., dogs, cats, and horses).

### 1. General Discussion of the Disclosed Embodiments

An understanding of the following basic chemical properties of amino acids is helpful to understand laboratory assay of the response to amino acids by The System.

Amino acids of interest include 1-3,4-dihydroxyphenylalanine (hereafter referred to as L-dopa) and 5-hydroxytryptophan (hereafter referred to as 5-HTP). L-dopa is an amino acid precursor of the catecholamine system (dopamine, norepinephrine, and epinephrine) and 5-HTP is an amino acid precursor of serotonin. Both share unique chemical properties in the human body and other higher forms of life, including:
1. Both can be absorbed into the system in significant amounts after oral ingestion.
2. Being water soluble both cross the blood brain barrier freely.
3. L-dopa is freely converted to dopamine and 5-HTP is freely converted to serotonin when without biochemical feed back regulation when exposed to the enzyme L-aromatic amino acid decarboxylase (also known as L-dopa decarboxylase or 5-HTP decarboxylase) which catalyzes the synthesis of both dopamine and serotonin in conjunction with required cofactors.
4. Neither is subject to biochemical regulation in the synthesis of dopamine and serotonin giving the unique ability to establish theoretical levels of dopamine and serotonin in unlimited amounts.

Other amino acid precursors include tryptophan of the serotonin system and include but are not limited to tyrosine, N-acetyl-1-tyrosine, and phenylalanine of the catecholamine systems. These amino acids share the same chemical properties as L-dopa and 5-HTP with the exception that they are subject to biochemical feedback regulation meaning that only a limited amount of dopamine and serotonin can be produced by the system with their administration. Production of tryptophan is regulated via the serotonin/tryptophan hydroxylase feedback loop and production of L-dopa from tryosine is regulated via the, norepinephrine/tyrosine hydroxylase feed back loop.

Laboratory assay of the serotonin and catecholamine systems can be carried out by different types and forms of assays of serum, saliva, urine, or any other method which accurately reflects the serotonin and dopamine levels of the system. Based on the following discussion, one method to measure or monitor serotonin and dopamine in the system is through the use of urinary assay.

With regards to serotonin and dopamine assay of serum, a major limitation is the collection of a sample. It is a fact that monoamine levels fluctuate greatly from minute-to-minute and the mere act of inducing a needle into a subject to obtain a serum or blood sample causes an instantaneous spike in the monoamine levels of the system which makes it impossible to obtain accurate level readings that are reflective of levels just prior to insertion of the needle. Methods to compensate for this limitation are cumbersome to the point of not being useful in routine evaluation of subjects. For example, to obtain a true baseline monoamine reading from serum the subject should have a central venous catheter inserted and be allowed to lie quietly in a darkened room for 30 minutes at which point a serum sample can be drawn as long as the subject is not disturbed. A second method of obtaining a serum sample that is meaningful in the assay of serum monoamine levels is to place an indwelling catheter (such as a hep-lock) in the subject and allow the subject several days to get used to the catheter at which point serum can be obtained. These two methods are not intended to be an exhaustive discussion on the methodology for obtaining valid serum specimens but are intended to illustrate the considerations that need to be made if serum assay of monoamine in support of amino acid administration is opted for.

Salivary assay may be an option but again saliva is subject to minute-to-minute variability of the system as a whole. A method to compensate for the fluctuations in the system is to obtain several (four to six) saliva samples during an approximate time period of 30 minutes then to average the results of the samples. The drawback of this method is that the final reported assay is a coarse approximation at best and the cost is higher since four to six independent tests need to be run, plus it requires specimen collection over a 30 minute period of time without interruption. To date, no accurate calibration of salivary monoamine testing has been perfected.

Another method is an assay of monoamine levels through urinary testing. This assay is not a completely straight forward assay either, and must be preformed with adherence to the following considerations. In reporting urinary assay results consideration must be made to compensate for dilution of the urine (specific gravity variance). Simply assaying the monoamines in a given urine sample will not give results of desired meaning due to variance in specific gravity from sample to sample. One method to compensate for variance in specific gravity is to report the results as a monoamine to creatinine ratio or other substance that shares the property of “constant excretion by the kidneys” with creatinine. One method reports results as micrograms of monoamine per gram of creatinine in the urine. In utilizing urinary laboratory assay of monoamines the problem of minute-to-minute spikes in the monoamine levels is overcome and the results reported are an average of the monoamine levels in the urine since the bladder was last emptied (generally 2 to 3 hours earlier). Other considerations of urinary monoamine assay include, but are not limited to: the urine should be collected 5 to 6 hours prior to onset of the sleep cycle although other times are available at the discretion of the individual initiating the test. This is contrary to the usual AM method for collection of urine for monoamine assay where a pathologic diagnosis of pheochromocytoma, or carcinoid syndrome is entertained. Urinary assay of monoamines in support of amino acid therapy of The System should be collected at or near the low point, 5 or 6 hours before bed time, to insure that a monoamine assay is obtained in an effort to ensure that monoamine levels do not drop below levels needed to keep the system free of disease symptoms (a therapeutic range), although collections at other times of the day may yield meaningful results which are less than optimal.

The kidneys uptake the serotonin and catecholamine as well as the amino acid precursors of the serotonin and catecholamine in the proximal convoluted tubules cells of the kidneys via the cation uptake ports found on the surface of the proximal convoluted renal tubule cells. The uptake of the serotonin and catecholamines and the amino acid precursors occurs after they are filtered by the glomeruli of the kidneys from the renal arterial blood flow. Once in the proximal convoluted renal tubule cells, the monoamines are metabolized into metabolites and the amino acids are synthesized into new monoamines as verified by radioisotope tagging studies. The term “therapeutic range” is defined as the concentration of monoamines where optimal neurotransmitter and regulatory functions occur. The primary application of laboratory assay of monoamines of The System is to assist in establishing therapeutic levels of monoamines, which correlate with the resolution of disease symptoms and/or optimized regulatory function. Discussed herein is one embodiment using 5-HTP as the component of the balanced administration of amino acids. Similar considerations exist for L-dopa and dopamine and other amino acid precursors of The System. In one embodiment, the daily milligram dosing is provided. In one embodiment, the urinary serotonin levels as reported in micrograms per gram of creatinine. In a subject who is treated with a significant quantity of balanced amino acids (use of amino acid precursors of both serotonin and catecholamine systems in combination) and has laboratory assay of monoamine performed frequently (with every 100 milligram increase in 5-HTP) the dose-response curve may show a phase 2 or a phase 3 response. The dose-response curve may also include phase 1, when a subject is treated with lower doses of amino acids and has laboratory assay of monoamine performed frequently (with every 100 milligram increase in 5-HTP). As amino acid dosing is increased, urinary monoamine levels decrease until the phase 2 response is established. In phase 2 response, urinary monoamine levels do not change and are flat as the amino acid dosing is increased until the inflection point is arrived at which the daily dosing of approximately 800 milligrams per day of 5-HTP, occurs for the phase 3 response. Once the inflection point is arrived at in the phase 3 response, small increases in the amino acid precursors dosing levels lead to large increases in the urinary monoamine levels. In recognizing this rapid upward inflection of the urinary monoamine levels the ability to define a therapeutic range in the treatment of monoamine dysfunction disease symptoms is gained. It is noted that the amino acid dosing of precursors needs of both the catecholamine and serotonin systems vary widely in a group of subjects with regards to dosing at which the inflection point occurs. The exact urinary monoamine levels of the optimal and therapeutic range may vary depending on the methodology of the laboratory doing the assay but in general the lower limits of the range need to be high enough to insure that symptoms of monoamine dysfunction are under control and the top end of the therapeutic range needs to be set at such a level as to insure that the subject is not being over loaded with monoamine during treatment leading to undesirable outcomes. In some embodiments, the therapeutic range for serotonin is set at 800 to 2400 micrograms of serotonin per gram of creatinine. Other ranges exist as discussed further. The three phase response will also be discussed further below.

In one embodiment, a first step may be to define a reference range via statistical analysis of the population to two standard deviations from the mean as is standard practice for laboratories. The respective laboratory reference range of serotonin may be defined as 100 to 250 micrograms of serotonin per gram of creatinine. It is recognized that many people with urinary monoamine assay values inside of the reference range are suffering from monoamine dysfunction related illness or regulatory problems, and the only way to provide effective relief of symptoms is to establish monoamine levels that are higher than the reference range in what is known as the therapeutic range. The Parkinson's disease model illustrates very well why levels higher than the reference range are needed. In many subjects, not just in Parkinsonism, monoamine levels higher than the reference range are needed to achieve results. But still there is a subgroup of people who have no symptoms of monoamine dysfunction and are functioning at a very high level. Use of monoamine assay in defining an optimal range for subjects who have no symptoms of monoamine dysfunction is another application of this invention.

While the discussion so far has focused on urinary neurotransmitter assay applications the following considerations are made for salivary, serum, or other test methods that reflect the monoamine status of the subject. With these other forms of assay there is also a flat dose-response curve in phase 2, to the point of inflection in phase 3, at which time small increases in amino acid dosing leads to large increases in the monoamines measured. With these other methods of assay there is also an optimal range that can be defined within the reference range.

All methods of assay when used properly insure that adequate amino acid precursors are given without overloading the system with monoamines. With all methods the response to a given dose of amino acids varies widely from subject to subject. For example, subjects have been seen who experience phase 2/phase 3 inflection point on 50 milligrams per day of 5-HTP, and others who do not experience the phase 2/phase 3 inflection point until 1,500 milligrams of 5-HTP per day is administered. When L-dopa and 5-HTP are used in combination for the synthesis of dopamine and serotonin respectively, to affect proper balanced administration of amino acids, monoamine levels of the system may be established that are well above the therapeutic range. When the other precursors of dopamine and serotonin are used, such as precursors of L-dopa and 5-HTP, in general, due to biochemical feed back regulation monoamine that are only near or in the lower end of the therapeutic range may be established, with a small probability of establishing monoamine levels that are it the higher end of the therapeutic range or higher than the therapeutic range.

The invention provides the ability to optimize group results in the treatment of The System related dysfunction via a safe and effective method to gain control of The System in the treatment of dysfunction, as well as to facilitate optimal function for systems dependant on the catecholamine and/or serotonin systems for regulation and function as guided laboratory assay of monoamine of The System. Laboratory values and amino acid dosing listed in this description are for obtaining optimal results in a human population. Adjustment in dosing for non-human populations should be made based on body weight and response as verified by laboratory assay.

A primary use of laboratory monoamine assay is three-fold:
1. To establish a baseline assay prior to treatment with amino acids.
2. To establish a therapeutic level of monoamines in treatment with amino acids whereby the monoamines are high enough in the system at the low point during the day to insure that symptoms of monoamine dysfunction are not present and that monoamine levels are not too high in the system so as to create other problems such as serotonin syndrome and the like.
3. To establish an optimal level of monoamines in those subjects not suffering symptoms of monoamine dysfunction.

In order to affect the three uses of monoamines described immediately above, monoamine assay via serum, saliva, urine, or other methods is used, as long as considerations of the limitations of each method as previously discussed are compensated for.

For saliva assay of monoamines of The System, a compensation is the need to perform several tests over a relatively short period of time (approximately 30 minutes) and averaging of the results.

For serum assay of monoamines of The System, a sample needs to be collected when the subject is not disturbed by needle puncture, etc, so as to not affect the baseline monoamine levels present just prior to collection of the sample.

For urinary assay of the monoamines of The System, a compensation is a method of reporting results whereby the variability in the specific gravity of the urine is compensated for.

Methods for compensation in saliva, serum, and urine were previously discussed and the following is a discussion of interpretation and applications of monoamine assay of monoamines of The System.

The following laboratory value numbers are for the specific laboratory used in the research of this invention. Due to variability in assay techniques between laboratories actual values may legitimately vary from laboratory to laboratory.

“REFERENCE RANGES” are the ranges set by the individual laboratory from statistical analysis of a population of subjects based on defining the mean and standard deviation. The typical reference range is the value found in calculating two standard deviations above and below the mean. The reference range reported by each laboratory may also be unique depending on the methodology of the assay being used. An exemplary embodiment of the reference range established by a first laboratory is as follows:

Serotonin=100 to 250 micrograms of monoamine per gram of creatinine.

Dopamine=100 to 250 micrograms of monoamine per gram of creatinine.

Norepinephrine=25 to 75 micrograms of monoamine per gram of creatinine.

Epinephrine=5 to 13 micrograms of monoamine per gram of creatinine.

Another exemplary embodiment of the reference range established by a second laboratory is as follows:

Serotonin=48.9 to 194.9 micrograms of monoamine per gram of creatinine.

Dopamine=40.0 to 390.0 micrograms of monoamine per gram of creatinine.

Norepinephrine=7.0 to 65.0 micrograms of monoamine per gram of creatinine.

Epinephrine=2.0 to 16.0 micrograms of monoamine per gram of creatinine.

OPTIMAL RANGES are defined as a narrow range within the reference range where subjects with no symptoms of monoamine dysfunction appear to be functioning optimally based on group observations. The optimal ranges for the monoamines of The System for the first laboratory above are as follows:

Serotonin=175 to 225 micrograms of monoamine per gram of creatinine.

Dopamine=125 to 175 micrograms of monoamine per gram of creatinine.

Norepinephrine=30 to 55 micrograms of monoamine per gram of creatinine.

Epinephrine=8 to 12 micrograms of monoamine per gram of creatinine.

The optimal ranges for the monoamines of The System for the second laboratory above are as follows:

Serotonin=85.6 to 175.4 micrograms of monoamine per gram of creatinine.

Dopamine=50.0 to 273.0 micrograms of monoamine r per gram of creatinine.

Norepinephrine=8.4 to 47.7 micrograms of monoamine per gram of creatinine.

Epinephrine=3.2 to 14.8 micrograms of monoamine per gram of creatinine.

THERAPEUTIC RANGES are the range to be obtained in treatment to insure that resolution of symptoms is affected without overloading the system on monoamines. The therapeutic ranges of the monoamines of The System are as follows. It should be noted that these numbers are a relative guide only for reaching an inflection point in treatment and that the therapeutic range should not be fixed on the absolute numbers reported. Instead, the therapeutic range is specific to the respective monoamine dysfunction disease. In general, the therapeutic range for serotonin in monoamine dysfunction is typically 800 to 2400 micrograms of monoamine per gram of creatinine and in phase 3. For example, the therapeutic range for serotonin disease is reported at 800 to 200.

In general, the therapeutic range for dopamine in monoamine dysfunction may vary from lab to lab and is typically 475 to 875 micrograms of monoamine per gram of creatinine.

Dopamine (in treatment of Parkinsonism) <20,000 micrograms of monoamine per gram of creatinine and treatment decisions are driven by clinical outcomes.

In general, the therapeutic range for norepinephrine in monoamine dysfunction is typically 7 to 65 micrograms of monoamine per gram of creatinine.

In general, the therapeutic range for epinephrine in monoamine dysfunction is typically 2 to 16 micrograms of monoamine per gram of creatinine.

The laboratory “reference range” is the values defined to within two standard deviations of the mean for the population tested while not under treatment. The therapeutic range is defined as being one to three standard deviations of the reference range above the high end of the reference range reported by the laboratory performing the assay. Under the laboratory reference range used in this invention the low end of the therapeutic range for urinary serotonin was defined as less than 800 micrograms of serotonin per gram of creatinine. Under the laboratory reference range used in this invention the low end of the therapeutic range for urinary dopamine was defined as less than 475 micrograms of serotonin per gram of creatinine.

The goal of treatment is to establish monoamine levels of The System in the optimal range for subjects with no symptoms of monoamine dysfunction and in the therapeutic range for subjects suffering from symptoms of monoamine dysfunction. To affect establishment of monoamine in the desired range, adjusting the dosing of amino acids is affected as described by the Applicant in U.S. Patent Application Publication No. U.S. 2003/0181509 A1, published Sep. 25, 2003, which is incorporated by reference in its entirety herein.

In certain embodiments, the dosing ratio of the amino acid precursors L-tyrosine and 5-HTP for optimal results is generally 10:1 on a milligram-to-milligram basis and the ratio of L-dopa to 5-HTP for optimal results when used is 1:3 or within 85% of these values as discussed in U.S. Patent Application Publication No. U.S. 2003/081509 A1, published Sep. 25, 2003. The dosing ratio of phenylalanine to 5-HTP for optimal results, when used, is 10:1; and the dosing ratio of N-acetyl-L-tyrosine to 5-HTP for optimal results, when used, is 5:1 or within 85% of these values as discussed in U.S. Patent Application Publication No. U.S. 2003/081509 A1, published Sep. 25, 2003. Other amino acid precursors of dopamine and serotonin may be used with considerations for ratios to obtain optimal results, but if the goal is optimal results with group treatment dosing ratios close (within 85%) to these should generally be used. Proper use of amino acid ratios is referred to as “balanced.”

Applications of the present invention relate to any application where the serotonin and/or catecholamine monoamine systems are involved. This includes treatment in and of itself in subjects suffering from central nervous system monoamine diseases, which includes, but is not limited to: depression, anxiety, panic attacks, migraine headache, obesity, bulimia, anorexia, premenstrual syndrome, menopause, insomnia, hyperactivity, attention deficit disorder (ADD), impulsivity, obsessionality, inappropriate aggression, inappropriate anger, psychotic illness, obsessive compulsive disorder (OCD), fibromyalgia, addictions, sexual dysfunction, chronic fatigue syndrome, chronic pain states, adrenal fatigue/burnout, attention deficit hyperactivity disorder (ADHD), Parkinsonism, traumatic brain injury, claustrophobia, tension headaches, nocturnal myoclonus, irritable bowel syndrome/Chron's disease, states of decreased cognitive function such as dementia and Alzheimer's disease, states associated with aging such as deterioration of organ systems innervated by the serotonin and/or catecholamine systems and deterioration of cognitive functions, hormone dysfunction problems innervated by the serotonin and/or catecholamine systems; cortisol dysfunction related problems, and monoamine reaction to chronic stress.

### 2. Description of Embodiments of the Invention

For monoamine testing of monoamines of The System, urine is collected approximately 5 to 6 hours prior onset of the sleep cycle and just prior to any amino acid dosing the subject may or may not be taking close to that time period. Once the urine sample is obtained, laboratory assay of the monoamine of The System are performed as well as a urinary creatinine assay and the results are reported in terms of micrograms of monoamine per gram of creatinine.

After there has been a start or change in the dosing of amino acid precursors of The System, the following considerations exist with regards to monoamine assay.
1. It takes 3 to 5 days for serotonin levels to come to equilibrium in the urine.
2. It takes 3 to 5 days for dopamine levels to come to equilibrium in the urine.
3. It takes 2 to 6 weeks for norepinephrine to come to equilibrium in the urine after the serotonin and dopamine have been established in the therapeutic range and in the phase 3 response.
4. It can take as long as 3 to 6 months for epinephrine to come to equilibrium in the urine after the serotonin and dopamine have established in the therapeutic range and in the phase 3 response.

If the subject is not under treatment with amino acid precursors of The System at the time the sample is collected, the results are used as a baseline reference point in treatment with amino acid precursors of The System.

If the subject has no symptoms of monoamine dysfunction the subject is treated with amino acids precursors of The System and the amino acid dosing increased or decreased as guided by laboratory assay of monoamines of The System until reported results of laboratory assay are in the optimal range.

If the subject is suffering from symptoms of monoamine dysfunction, the subject is treated with amino acid precursors of The System which are in turn increased or decreased until urinary monoamine levels of The System have been established in the therapeutic range.

Retesting of the subject should be done one week or more after every dose change in the amino acid precursors takes place.

Once under treatment and optimal or therapeutic ranges have been established periodic retesting should be preformed at regular intervals. It is the preferred method to perform follow up testing every six months or sooner.

If assay of monoamine reveals that the urinary monoamine levels are low, the correct response is to increase the amino acid precursor dosing of The System. If assay of the monoamines reveals that the urinary monoamine levels are high the correct response is to decrease the amino acid precursor dosing of The System.

With the addition of unopposed L-dopa the urinary excretion of serotonin in phase 3 increases markedly, a fact that was not previously known. The same is true with administration of unopposed precursors of the serotonin system such as 5-HTP. With the administration of 5-HTP alone there is a marked increase in the excretion of dopamine in phase 3 by the kidneys. These observations are of importance in establishing optimal and therapeutic ranges of monoamines as guided by laboratory assay. For example, in a subject under treatment with amino acid precursors of The System who is experiencing symptoms of monoamine dysfunction and has a dopamine reported by assay of 150 micrograms of dopamine per gram of creatinine and a serotonin of 1,100 micrograms of serotonin per gram of creatinine, and is still experiencing symptoms of monoamine dysfunction, management considerations are as follows.
1. By giving more of the balanced amino acid precursor of The System the urinary levels of both serotonin and dopamine levels will rise. This may lead to resolution of symptoms as the dopamine levels are established in the therapeutic range but the serotonin levels will be higher than the therapeutic or optimal range as well.
2. By administering only amino acid precursors of dopamine the ability is gained to establish dopamine levels in the therapeutic or optimal ranges. Using unopposed amino acid precursors in treatment, the excretion of serotonin by the kidneys increases markedly again leading to a situation where the dopamine is in the therapeutic range and the urinary serotonin levels are higher than the therapeutic or optimal ranges.
3. The proper management of a subject with 150 micrograms of dopamine per gram of creatinine and 1,100 micrograms of serotonin per gram of creatinine level is to increase the amino acid precursors of dopamine while at the same time decreasing the amino acid precursors of serotonin to give an outcome whereby monoamines of both systems are in the therapeutic or optimal ranges.

Not all monoamine dysfunction related symptoms resolve in the same therapeutic ranges. In general treatment of obesity, panic disorder, and obsessive compulsive disorder require urinary serotonin levels of 250 to 1,200 micrograms of serotonin per gram of creatinine. For diseases other than obesity, obsessive compulsive disorder, and panic disorder, serotonin levels of 1,200 to 2,400 micrograms of serotonin per gram of creatinine are the usual range. In the treatment of Parkinsonism the therapeutic range for serotonin is 250 to 2,400 micrograms per gram of creatinine and the therapeutic range is to elevate the dopamine levels high enough to get the symptoms of Parkinsonism under control. In general, in treatment of Parkinsonism the therapeutic range is to keep the dopamine levels less than 20,000 micrograms of dopamine per gram of creatinine. Although preferred numbers for a therapeutic range are provided here, it should be understood that variance in lab techniques may change these numbers and that testing should be used as a guide to insure that the phase 3 inflection point has been reached without overloading the system. It should be understood that recognition of an inflection point during treatment is an important consideration.

Simply establishing urinary monoamine levels in the therapeutic range may not control symptoms in all subjects. For example, in treatment of the obese subject with a urinary serotonin assay of 2,300 micrograms of serotonin per gram of creatinine and a dopamine level of 500 micrograms of dopamine per gram of creatinine with the norepinephrine and epinephrine levels in the therapeutic ranges as well and the subject is not losing weight considerations are as follows. In a case such as this from a monoamine standpoint of The System, further treatment may be limited and other causes that are preventing the subject from losing weight should be considered. In most cases such as this, there is a major stressor in the subject's life that can be identified which is distracting them from doing the things they need to do to be successful at weight loss. Considerations such as this also apply to other monoamine dysfunction symptoms of illness.

Referring now to the data summarized herein, the data is from a database of subjects who have been diagnosed by a licensed healthcare provider as having one or more monoamine dysfunction(s) associated with the serotonin and/or catecholamine systems. The database fields include:
1. The monoamine diagnosis made by a licensed healthcare provider;
2. Amino acid dosing of amino acid precursors of the serotonin and dopamine monoamines;
3. Laboratory assay of urinary serotonin and catecholamine monoamine levels;
4. The subject's sex;
5. The subject's age and date of birth;
6. The subject's weight;
7. The subject's height;
8. Any allergies the subject might have;
9. Any medications (prescription and/or non-prescription) with dosing that the subject might be taking;
10. Type of license held by the caregiver; and
11. The subject's state and zip code of residence.

Also referring to the summarized date, the monoamine analysis performed was subjected to the following criteria:
1. Urinary monoamine reference ranges reported by the laboratory were determined after precision and accuracy studies had validated initial calibration results;
2. The reported reference ranges of the laboratory performing the assays were within ±2 standard deviations of the mean for the general population used in calibration;
3. The reported reference ranges are for adults (18 years of age and older);
4. Urinary monoamine samples were collected at least 3 to 4 hours after a subject took a dose of amino acid precursors;
5. Urinary monoamine samples were collected 5 to 6 hours before the onset of the sleep cycle (bed time);
6. Urinary monoamine samples were only collected from subjects who had taken the amino acid precursors of the serotonin and dopamine monoamines consistently for 7 days or more;
7. The monoamines in the urinary sample were stabilized between collection and assay with 6N hydrochloric acid; and
8. Urinary dopamine, norepinephrine, and epinephrine monoamine levels were assayed by Radioimmunoassay (RIA) and urinary serotonin monoamine levels were assayed by ELISA.

Assay results are summarized of urinary serotonin, dopamine, norepinephrine, and epinephrine monoamine levels of subjects who were not taking any supplemental amino acid precursors and who also were diagnosed with one or more monoamine dysfunction diseases by a licensed healthcare provider.

Results of two urinary monoamine assays of serotonin and catecholamine levels are summarized for 65 subjects who were diagnosed with one or more monoamine dysfunction diseases by a licensed healthcare provider. The first assay was performed while the subjects were not taking any supplemental amino acid precursors of serotonin and dopamine, and the second assay was performed with the subjects taking supplemental amino acid precursors of serotonin and dopamine for at least seven days. The percentage of subjects who had serotonin and catecholamine monoamine levels higher than the laboratory reference range when not taking any supplemental amino acid precursors of serotonin and dopamine (first assay) and when the subjects were taking supplemental amino acid precursors of serotonin and dopamine (second assay).

The observed changes between the first and second assay can be grouped into four categories: (i) serotonin monoamine levels increased between assays; (ii) serotonin monoamine levels decreased between assays; (iii) dopamine monoamine levels increased between assays; and (iv) dopamine monoamine levels decreased between assays. In the first group, serotonin monoamine levels increased between assays, an increase in urinary serotonin monoamine levels is the independent variable between assay 1 and assay 2. In the second group, serotonin monoamine levels decreased between assays, a decrease in urinary serotonin monoamine levels is the independent variable between assay 1 and assay 2. In the first and second group, the dependent variables reported include changes in urinary dopamine monoamine levels between assay 1 and assay 2, an increase in 5-HTP amino acid dosing between assay 1 and assay 2, and an increase in tyrosine dosing between assay 1 and assay 2. In the third group, dopamine monoamine levels increased between assays, an increase in urinary dopamine monoamine levels is the independent variable between assay 1 and assay 2. In the fourth group, dopamine monoamine levels decreased between assays, a decrease in urinary dopamine monoamine levels is the independent variable between assay 1 and assay 2. In the third and fourth group, serotonin the dependent variables reported include changes in urinary serotonin monoamine levels between assay 1 and assay 2, an increase in 5-HTP amino acid dosing between assay 1 and assay 2, and an increase in tyrosine dosing between assay 1 and assay 2.

From the statistical analysis, there appears to be no predictability in monoamine level outcomes in changing amino acid doing in subjects taking amino acid precursor combinations of serotonin and dopamine where the dosing of the precursors is changed between assay 1 and assay 2 (N =65). The statistical analysis reveals the following between assay 1 where subjects were taking no amino acid precursors, and assay 2, where subjects were taking supplemental amino acid precursors of serotonin and dopamine:
1. In subjects where urinary serotonin increased between assays, 61% of the subjects had decreased dopamine monoamine levels while 39% of the subjects had increased dopamine monoamine levels;
2. In subjects where urinary serotonin decreased between assays, 62.5% of the subjects had increased dopamine monoamine levels while 37.5% of the subjects had decreased dopamine monoamine levels;
3. In subjects where urinary dopamine increased between assays, 51.6% of the subjects had increased serotonin monoamine levels while 48.4% of the subjects had decreased serotonin monoamine levels; and
4. In subjects where urinary dopamine decreased between assays, 73.5% of the subjects had increased serotonin monoamine levels while 26.5% of the subjects had decreased serotonin monoamine levels.

The statistical analysis and the preceding discussion illustrates that the predictability of determining an individual or group response of urinary monoamine levels between a first assay, where the subject is not taking any supplemental amino acid precursors of the serotonin and dopamine monoamines, and a second assay, where the subject is taking supplemental amino acid precursors of the serotonin and dopamine monoamines, is very poor.

Results of two urinary monoamine assays of serotonin and catecholamine levels are summarized of subjects who were diagnosed with one or more monoamine dysfunction diseases by a licensed healthcare provider. The first assay was performed while the subjects were taking supplemental amino acid precursors of serotonin and dopamine. The second assay was performed after the subject's supplemental amino acid precursor dosing of serotonin and dopamine were changed to move the urinary serotonin and dopamine monoamine levels closer to the ranges of 300 to 600 micrograms of dopamine per gram of creatinine and 800 to 2400 micrograms of serotonin per gram of creatinine and in the third phase.

A statistical analysis of 151 subjects where the dosing of both amino acid precursors (5-HTP and L-Tyrosine) of the serotonin and dopamine monoamines were decreased between the first and second assay. As summarized, 27.2% (N=41) of the subjects had increased serotonin monoamine levels while 78.8% (N=110) of the subjects had decreased serotonin monoamine levels; and 62.2% (N=94) of the subjects had increased dopamine monoamine levels while 37.7% (N=57) of the subjects had decreased dopamine monoamine levels.

A statistical analysis of 25 subjects where the dosing of amino acid precursor (5-HTP) of the serotonin monoamine was decreased and the dosing of the amino acid precursor (L-Tyrosine) of the dopamine monoamine was increased between assays. As summarized, 20.0% (N=5) of the subjects had increased serotonin monoamine levels while 80.0% (N=20) of the subjects had decreased serotonin monoamine levels; and 48.0% (N=12) of the subjects had increased dopamine monoamine levels while 52.0% (N=13) of the subjects had decreased dopamine monoamine levels.

A statistical analysis of 6 subjects where the dosing of amino acid precursor (H-HTP) of the serotonin monoamine was increased and the dosing of the amino acid precursor (L-Tyrosine) of the dopamine monoamine was increased between assays. As summarized, 50.0% (N=3) of the subjects had increased serotonin monoamine levels while 50.0% (N=3) of the subjects had decreased serotonin monoamine levels; and 33.3% (N=2) of the subjects had increased dopamine monoamine levels while 66.7% (N=4) of the subjects had decreased dopamine monoamine levels.

A statistical analysis of 196 subjects where the dosing of both amino acid precursors (5-HTP and L-Tyrosine) of the serotonin and dopamine monoamines were increased between assays. As summarized, 65.8% (N=67) of the subjects had increased serotonin monoamine levels while 34.2% (N=129) of the subjects had decreased serotonin monoamine levels; and 49.0% (N=96) of the subjects had increased dopamine monoamine levels while 51.0% (N=100) of the subjects had decreased dopamine monoamine levels.

A statistical analysis of 40 subjects where the dosing of the amino acid precursor (5-HTP) of the serotonin monoamine was not changed and the dosing of the amino acid precursor (L-Tyrosine) of the dopamine monoamine was increased between assays. As summarized, 65.0% (N=26) of the subjects had increased serotonin monoamine levels while 35.0% (N=14) of the subjects had decreased serotonin monoamine levels; and 40.0% (N=16) of the subjects had increased dopamine monoamine levels while 60.0% (N=24) of the subjects had decreased dopamine monoamine levels.

The preceding discussion illustrates that the predictability of determining an individual or group response of urinary monoamine levels between two assays, where the subject is taking a dosing of supplemental amino acid precursors of the serotonin and dopamine monoamines, and the dosing is changed between the two assays, is very poor.

However, according to certain embodiments, the predictability of determining an individual or group response of urinary serotonin and/or dopamine monoamine levels between two assays is possible when it is properly determined that the subject is in one of three phases of urinary monoamine response to amino acid precursor administration. As identified above, there are at least three phases (referred to as phase 1, phase 2, and phase 3) of urinary monoamine response to amino acid precursor administration. In phase 1, the urinary monoamine level drops as the total amino acid precursor dosing of serotonin and dopamine is increased. In phase 1, the urinary monoamine level increases as the total amino acid precursor dosing of serotonin and dopamine is decreased. In phase 2, the urinary monoamine level remains below the therapeutic range and relatively constant as the total amount of monoamine amino acid precursor dosing is increased or decreased. In order for urinary monoamines to be in phase 2, subtherapeutic urinary monoamine levels must exist. In phase 3, the urinary monoamine level increases as the total amount of amino acid precursor dosing is increased. In phase 3, the urinary monoamine level decreases as the total amount of amino acid precursor dosing is decreased.

In order to obtain optimal results, typically the urinary serotonin and dopamine levels must be in the therapeutic range and in the third phase. Once the urinary serotonin and dopamine levels are in the therapeutic range and in the third phase, it may take 2 to 6 weeks for the norepinephrine to equilibrate and 3 to 6 months for the epinephrine to equilibrate (although in a limited number of cases it has been observed that it can take as long as 8 to 9 months for the epinephrine to equilibrate). This phenomenon has been observed in all other life forms tested where the serotonin and catecholamine systems with renal systems exist (i.e., cats, dogs, and horses, etc.).

As previously discussed, the amino acid precursors of the serotonin system must be in balance with the amino acid precursors of the catecholamine system to optimally treat monoamine dysfunction. While previous teachings indicated that a ratio of the amino acid precursor of the serotonin system and the catecholamine system were needed for optimal results, which was typically within 85% of a 10 to 1 ratio of the dopamine to serotonin amino acid precursor, in some embodiments the proper use of laboratory testing allows for individual refinement of the ratio between the serotonin and catecholamine precursors allowing for improved outcomes.

In order to achieve the necessary refinement of the ratio between the serotonin and catecholamine precursors for the treatment of monoamine dysfunction, an understanding of the basic laboratory testing results that are observed with measurement of urinary monoamines is necessary.

In certain embodiments, the phase of the urinary monoamines in response to amino acid therapy can properly be determined by obtaining two laboratory assays on two separate days with the subject taking a different amino acid dosing of serotonin and catecholamine precursors for each test. In general, at least 7 days must elapse between amino acid dosing changes and laboratory assay, but shorter time periods may be indicated at the decision of the individual obtaining the test. The laboratory assays should also be performed on samples that are collected at least 3 to 5 days after the last change in amino acid precursor dosing or start in amino acid precursor dosing of the serotonin and dopamine monoamines. If during the first assay the subject was taking supplemental amino acid precursors of serotonin and dopamine monoamines, then it is preferable that the second assay is performed after the dosing of the supplemental amino acid precursors has changed. The change in supplemental amino acid precursor may be for the serotonin monoamine and/or the dopamine monoamine. If both of the supplemental amino acid precursors of the serotonin and dopamine monoamines are changed, it is preferable that the dosing of both supplemental amino acid precursors is increased or the dosing of both supplemental amino acid precursors is decreased. The subject also should have not missed any dosing of amino acid precursors in the 3 to 5 day period before the sample is collected.

In order to obtain urinary serotonin and dopamine levels in the therapeutic range, which relieves a significant amount of symptoms in patients, optimal results are only typically obtained when the urinary serotonin and dopamine are in “the therapeutic range and both in the phase 3 response.” Once the urinary serotonin and dopamine are in the therapeutic range and in the phase 3 response, conditions are arrived at that correlate highly with resolution of disease symptoms, optimal feeling of wellness and well being, optimal individual outcomes, and optimal group outcomes.

A relief of monoamine dysfunction symptoms may also be obtained during the process of moving the urinary serotonin and dopamine monoamine levels in balance toward the therapeutic range and phase three response without actually reaching the therapeutic range and phase three response. For example, it has been observed that a subject suffering from migraines has obtained relief of symptoms once the urinary monoamine levels of serotonin and dopamine were less than 1000 and 500 micrograms of monoamine per gram of creatinine, respectively, even though the subject's dopamine was still in the phase 1 response with a serotonin in the phase 3 response. While the relief of symptoms associated with monoamine dysfunction may be observed in any of the three phases, the administration of amino acid precursors of serotonin and dopamine in balance as guided by laboratory assay is a very useful technique and therapy to establish urinary serotonin and dopamine levels that are in the therapeutic range and in the phase 3 response, which are the key to optimal outcomes.

In certain embodiments, as the amino acid dosing of balanced amino acid precursors are changed, the urinary serotonin and dopamine monoamine level results discussed below may be observed. These laboratory assay results will guide the proper treatment needed in order to alleviate the inappropriate excretion of urinary serotonin and catecholamine monoamines. These laboratory assay results will also guide the proper treatment needed to get both of the urinary serotonin and dopamine monoamine levels into the therapeutic range and in the phase 3 response.

In subjects diagnosed as suffering from one or more monoamine diseases and regulatory dysfunction and who have had a first laboratory assay performed after taking a first dosing of amino acid precursors of serotonin and dopamine monoamines and then have had a second laboratory assay performed after the amino acid precursor dosing has changed, three distinct phase responses exist, and are explained as follows:

1. Phase 1:
a. Where an increase in amino acid precursor dosing of either the serotonin amino acid precursor and/or the dopamine amino acid precursor causes a decrease in the second assay urinary serotonin or dopamine monoamine levels.
b. Where a decrease in amino acid dosing of either the serotonin amino acid precursor and/or the dopamine amino acid precursor causes an increase in the second assay urinary serotonin or dopamine monoamine levels.

2. Phase 2:
a. Where urinary serotonin and/or dopamine monoamine levels are less than the therapeutic range.

3. Phase 3:
a. Where an increase in amino acid precursor dosing of either the serotonin amino acid precursor and/or the dopamine amino acid precursor causes an increase in the second assay urinary serotonin or dopamine monoamine levels.
b. Where a decrease in the amino acid precursor dosing of either the serotonin amino acid precursor and/or the dopamine amino acid precursor causes a decrease in the second assay urinary serotonin or dopamine monoamine levels.

In certain embodiments, the amino acid dosing range needed to move urinary serotonin or dopamine from phase 1 to phase 3 also varies widely. For example, one observed subject who was in phase 1 response only required an amino acid dosing increase of the serotonin precursor by 150 mg to move from phase 1 to phase 3, with similar results observed for dopamine and its precursors. In contrast, other observed subjects required an amino acid dosing increase of the serotonin precursor by 2,000 mg to move from phase 1 to phase 3, with similar results observed for dopamine and its precursors. For example, some observed subjects moved between phase 1 and phase 3 with very small amino acid dosing changes (37.5 mg of 5-HTP with 375 mg of L-Tyrosine or 60 mg or L-Dopa) while other subjects required much larger amino acid dosing changes (900 mg of 5-HTP with 5,000 mg of L-Tyrosine or 900 mg of L-Dopa).

In certain embodiments, the last dosing of amino acid precursors of the serotonin and catecholamine systems also varies widely. The goal of adjusting the amino acid precursor dosing between assays is to obtain an outcome where the urinary dopamine was in the phase 3 response with a level of 300 to 600 micrograms of dopamine per gram of creatinine, and the urinary serotonin was also in the phase 3 response with a level of 800 to 2400 micrograms of serotonin per gram of creatinine. Other ranges within that range are also contemplated. In one embodiment, the range for the last dosing of the 5-HTP amino acid precursor of the serotonin monoamine has been observed to be between 37.5 mg and 1500 mg while the last dosing of the L-Tyrosine amino acid precursor of the dopamine monoamine has been observed to be between 375 mg and 8250 mg. In certain embodiments, the preferred daily amino acid dosing in the therapeutic range and in the phase 3 response is 300 mg to 900 mg of 5-HTP and 5000 mg to 8000 mg of L-Tyrosine. Other ranges within each of those ranges are also contemplated.

In certain embodiments, the dosing needs of the amino acid precursor of serotonin are independent of the dosing needs of the amino acid precursor of dopamine in adjusting the amino acid precursors to obtain a phase 3 response for both the serotonin and dopamine monoamines. By increasing the precursor of only one system (either serotonin precursor or catecholamine precursor) the other system may also be affected and show changes that are found on laboratory assay.

If on any single test the urinary serotonin or dopamine is below the therapeutic range the serotonin or dopamine is in phase 2. In comparing two tests if urinary serotonin or dopamine is below the therapeutic range this take precedence to variability reported in the monoamine to creatinine ratios reported and both are simply in phase 2.

If urinary serotonin or dopamine is greater than the low end of the therapeutic range defined for the monoamine it is in either in phase 1 or phase 3.

The urinary serotonin or dopamine phases are independent of each other. For example with the phase of serotonin or dopamine in any of the three phases the phase of the other may be in any of the three phases with the exception that serotonin and dopamine do not both exist in phase 1 simultaneously.

If there is an increase in the total amino acid precursor dosing of serotonin and dopamine between the first assay and the second assay that is associated with an decrease in concentration urinary serotonin and/or dopamine monoamine to creatinine ratio between the first and second assay where both assays reveal monoamine to creatinine ratios for the respective monoamine that are greater than the low end of the therapeutic range for the associated monoamine the reported values of urinary serotonin or dopamine in are in phase 1.

In cases where a large increase in amino acid precursor dosing is given between the first and second assay with the serotonin or dopamine in phase 1 on the first assay and phase 3 as the urinary serotonin or dopamine moves from phase 1 on the first assay, through phase 2, then into a phase 3 on the second assay, on the second assay the monoamine creatinine ratio reported by the lab may actually increase or decrease on a relatively small level with a higher or lower phase 3 monoamine creatinine ratio reported than the initial phase 1 monoamine creatinine ratio reported.

If there is an increase in the total amino acid precursor dosing of serotonin and dopamine between the first assay and the second assay that is associated with an decrease in concentration urinary serotonin and/or dopamine monoamine to creatinine ratio between the first and second assay, where the first assay reveals monoamine to creatinine ratio for the respective monoamine that is greater than the low end of the therapeutic range, and the second assay reveals monoamine to creatinine ratio that is less than the low end of the therapeutic range for the associated monoamine, the reported values of urinary serotonin or dopamine in are in phase 1 on the first test and in phase 2 on the second test.

If there is an increase in the total amino acid precursor dosing of serotonin and dopamine between the first assay and the second assay that is associated with an increase in concentration urinary serotonin and/or dopamine monoamine to creatinine ratio between the first assay and the second assay, where the second assay reveals monoamine to creatinine ratios for the respective monoamine that are greater than the low end of the therapeutic range, and the first assay reveals monoamine to creatinine ratio that is less than the low end of the therapeutic range for the associated monoamine, the reported values of urinary serotonin or dopamine in are in phase 2 on the first assay and in phase 3 on the second assay.

If there is an increase in the total amino acid precursor dosing of serotonin and dopamine between the first assay and the second assay that is associated with an increase in concentration urinary serotonin and/or dopamine monoamine to creatinine ratio between the first and second assay, where both assays reveal monoamine to creatinine ratios for the respective monoamine that are greater than the low end of the therapeutic range for the associated monoamine, the reported values of urinary serotonin or dopamine in are in phase 3.

If there is an decrease in the total amino acid precursor dosing of serotonin and dopamine between the first assay and the second assay that is associated with an decrease in concentration urinary serotonin and/or dopamine monoamine to creatinine ratio between the first and second assay, where both assays reveal monoamine to creatinine ratios for the respective monoamine that are greater than the low end of the therapeutic range for the associated monoamine, the reported values of urinary serotonin or dopamine in both assays are in phase 3.

In cases where a large decrease in amino acid precursor dosing is given between the first and second assay with the serotonin or dopamine in phase 3 on the first assay and phase 1 as the urinary serotonin or dopamine moves from phase 3 on the first assay, through phase 2, then into a phase 1 on the second assay, on the second assay the monoamine creatinine ratio reported by the lab may actually increase or decrease on a relatively small level with a higher or lower phase 1 monoamine creatinine ratio reported than the initial phase 3 monoamine creatinine ratio reported.

If there is an decrease in the total amino acid precursor dosing of serotonin and dopamine between the first assay and the second assay that is associated with an decrease in concentration urinary serotonin and/or dopamine monoamine to creatinine ratio between the first and second assay, where the first assay reveals monoamine to creatinine ratio for the respective monoamine that is greater than the low end of the therapeutic range and the second assay reveals monoamine to creatinine ratio that is less than the low end of the therapeutic range for the associated monoamine, the reported values of urinary serotonin or dopamine in are in phase 3 on the first test and in phase 2 on the second test.

If there is an decrease in the total amino acid precursor dosing of serotonin and dopamine between the first assay and the second assay that is associated with an increase in concentration urinary serotonin and/or dopamine monoamine to creatinine ratio between the first assay and the second assay, where the second assay reveals monoamine to creatinine ratios for the respective monoamine that are greater than the low end of the therapeutic range and the first assay reveals monoamine to creatinine ratio that is less than the low end of the therapeutic range for the associated monoamine, the reported values of urinary serotonin or dopamine in are in phase 2 on the first assay and in phase 1 on the second assay.

If there is an decrease in the total amino acid precursor dosing of serotonin and dopamine between the first assay and the second assay that is associated with an increase in concentration urinary serotonin and/or dopamine monoamine to creatinine ratio between the first and second assay, where both assays reveal monoamine to creatinine ratios for the respective monoamine that are greater than the low end of the therapeutic range for the associated monoamine, the reported values of urinary serotonin or dopamine in both assays are in phase 1.

While the phase illustrations just put forth are meant as examples of how the phase a monoamine can be determined. The phases of both urinary serotonin and dopamine need to be determined and the phase of each exists independent of the phase of the other.

### 3. Examples

### Example 1

A subject administered an increase in the dopamine precursor tyrosine caused a marked increase in urinary serotonin levels on the second laboratory assay without much change in the urinary dopamine levels which as subtherapeutic. In this case, the difference between the first and second laboratory assays reveals that the urinary serotonin levels were in phase 3 and the urinary dopamine levels were in phase 2. As previously discussed above, the phase of urinary serotonin and dopamine levels can be ascertained by administering or taking away only one precursor of serotonin or dopamine in a patient under treatment with balanced amino acids.

### Example 2

A subject administered an increase in the dopamine precursor L-dopa, between the first and third assay, increased the urinary serotonin level. This series of three tests also demonstrates a very narrow dosing range between phase 1 and phase 3 for urinary dopamine levels. In analyzing all 3 laboratory assays, the first test revealed urinary dopamine levels in phase 1, the second test revealed the urinary dopamine levels to be unknown, and the third test revealed urinary dopamine levels in phase 3. Similar results have been observed with urinary serotonin levels, which can also have a very narrow range between phase 1 and phase 3. As previously discussed, the phase of urinary dopamine or serotonin levels can be established by use of either serotonin and/or dopamine precursors in conjunction with laboratory assay.

### Example 3

The first and second laboratory assays reveal a urinary serotonin level in phase 1. The third laboratory assay reveals urinary serotonin levels in phase 2, and the fourth laboratory assay reveals a phase 3 response. The urinary dopamine levels are in phase 2 on the first three tests and in phase 3 on the third test. It is the teaching of this invention, which was previously discussed above, that the urinary serotonin and dopamine levels may be in different phases as serotonin and dopamine are independent of each other.

### Example 4

Adding precursors of one system can decrease the precursor needs of the other system for establishing urinary monoamine level in the therapeutic range and the phase 3 response. In examining the administered laboratory assay results, test 1 reveals a phase 3 response for the urinary serotonin levels, test 2 reveals a therapeutic urinary serotonin level with the phase unknown, and test 3 reveals a phase 3 urinary serotonin level that is high (above the therapeutic range). The first 3 laboratory assays results reveals that the phase 3 therapeutic range for urinary serotonin levels is between 300 mg and 900 mg per day of 5-HTP. As the dopamine precursor tyrosine is increased, the final laboratory assay results reveal a phase 3 therapeutic urinary serotonin level with a 5-HTP dosing of 150 mg per day. As demonstrated from these laboratory assay results, the serotonin precursor needs of 5-HTP are ½ to ¼ of that which were needed with tyrosine dosing at lower levels.

### Example 5

A urinary serotonin level that is in phase 3 on the first laboratory assay result and then in a subtherapeutic range on the second test. Laboratory assays tests 3 and 4 reveals the urinary serotonin levels in phase 3. The urinary dopamine levels are in phase 2 on the first test, phase 1 on the second test, phase 2 on the third test, and finally into phase 3 therapeutic range on the last test.

### Example 6

Example 6 related to a subject with a very narrow amino acid dosing range between phase 1 and phase 3. In the first assay the serotonin levels were in phase 3, in the second assay the serotonin levels were in phase 1, and in the third assay the serotonin levels were in phase 2. In this example, the change of 5-HTP dosing by 150 mg per day between test 1 and test 2 moved the urinary serotonin levels from phase 3 to phase 1.

It certain embodiments, optimal results are obtained when the dosing of amino acid precursors of serotonin and dopamine monoamines are changed in such a manner that one of the precursors of serotonin or dopamine dominates the change (address primarily one system at a time even though changes will be appreciated in both systems).

In some embodiments, the monoamine transporter system serotonin, dopamine and their amino acid precursors found in the filtrate from the glomerulous enter the proximal convoluted tubules in the dilute urine. Proximal convoluted tubules are lined by “proximal convoluted renal tubule cells”. The organ cation transporters (OCT) of the proximal convoluted renal tubules cells transport serotonin, dopamine and amino acid precursors from the proximal convoluted tubules into the proximal convoluted renal tubule cells.

Serotonin and dopamine metabolism and/or synthesis from amino acid precursors occur between the peripheral blood and the urine. These processes follow the following course, which ultimately leads to serotonin and/or dopamine secretion into the renal vein or excretion into the final (concentrated) urine.

Metabolism of monoamines, path to urine.
a. renal artery.
b. afferent arteriole.
c. glomerulus.
d. bowman's capsule.
e. proximal convoluted tubules.
f proximal convoluted renal tubule cells.
g. Metabolized by the monoamine oxidase in the proximal convoluted renal tubule cells.
h. Metabolites excreted back into the proximal convoluted tubule.
i. Process while traveling to the final urine.

Amino acid precursor pathway until synthesized into new monoamines.
a. Renal artery.
b. Afferent arteriole
c. Glomerulus
d. Bowman's capsule
e. Proximal convoluted tubules
f Proximal convoluted renal tubule cells
g. Synthesized by L-aromatic amino acid decarboxylase (AAAD) enzyme to new monoamines.

Once synthesized the new monoamines meet one of two fates.
a. transported by the basolateral transporter of the proximal convoluted renal tubule cells to the interstitium then onto the renal vein.
b. Transported by the apical transporter of the proximal convoluted renal tubule cells to the proximal convoluted renal tubule then onto the final urine.

The monoamine oxidase (MAO) metabolizes serotonin and dopamine taken up from the proximal renal tubules in the proximal convoluted renal tubules cells. Uptake and metabolism is so efficient that very little of the serotonin and dopamine filtered at the glomerulous is found in the final concentrated urine.

Enzymatic activity of the monoamine oxidase (MAO) and the catecholamine-O-methyl transferase (COMT) facilitates the metabolism of serotonin and dopamine taken up by the proximal convoluted renal tubules cells organic cation transporters. This metabolism of by the MAO and COMT is subject to competitive inhibition between serotonin and dopamine. The uptake and metabolism is very efficient, which results in very little of the serotonin and dopamine filtered at the glomerulous in the final concentrated urine.

The serotonin and dopamine amino acid precursors taken up at the organic cation transporters (OCT1) are synthesized into serotonin and dopamine by the enzymatic action of the aromatic-L-amino aid decarboxylase (AAAD) enzyme. There is competitive inhibition between the amino acid precursors in synthesis when significant levels of amino acid precursors are being administered.

The amino acid precursors of serotonin and dopamine taken up from the proximal renal tubules by the organic cation transporters of the proximal convoluted renal tubule cells are synthesized into new serotonin and dopamine by the aromatic-L-amino aid decarboxylase (AAAD) enzyme. This newly synthesized serotonin and dopamine is then transported out of the proximal convoluted renal tubule cells by either the “basolateral monoamine transporter” or the “apical transporter”. The basolateral monoamine transporter is conceptualized as dominating this process as an organic cation transporter (OCT). The apical transporter is conceptualized as transporting serotonin and dopamine not transported by the basolateral monoamine transporter to the urine.

The amino acid precursors of serotonin and dopamine undergo uptake by organic cation transporters into the proximal renal tubules in a competitive manner. Administration of unbalanced amino acid precursors, where the dopamine precursors dominate, will inhibit the uptake of serotonin precursors leading to a decrease in serotonin synthesis. Administration of unbalanced precursors dominated by serotonin precursors will inhibit uptake of dopamine precursors leading to a decrease in dopamine synthesis. Properly balanced serotonin and dopamine amino acid precursors must be administered in an adequate amount to affect optimal synthesis. Imbalanced or inadequate administration of the amino acid precursor of serotonin or dopamine will lead to an imbalance or suboptimal synthesis of serotonin or dopamine contributing to depletion of the non-dominant monoamine.

Serotonin and dopamine exists in two states. The endogenous state is found when no supplemental amino acid precursors are administered. The competitive inhibition state is found when significant amounts of both serotonin and dopamine amino acid precursors are administered. The competitive inhibition state is of consideration in synthesis, metabolism, and transport of serotonin and dopamine. In at least one embodiment, this invention is directed to a method to objectively place serotonin and dopamine in the competitive inhibition state and then to manipulate one or both for optimum results.

Serotonin and dopamine are synthesized by the L-aromatic amino acid decarboxylase enzyme (AAAD). If amino acid precursor administration is out of balance then the dominant precursor will compromise synthesis of the other monoamine, leading to depletion and imbalance of the non-dominant monoamine.

In the competitive inhibition state a high level of one precursor leading to a dominant level of serotonin or dopamine will increase monoamine oxidase activity causing increased metabolism and depletion of the non-dominant monoamine.

In the competitive inhibition state a high level of one precursor will lead to a high level of the monoamine associated with the precursor. In the process the transport of the amino acid precursor and monoamine of the non-dominant system will be compromised. Transport being an integral part of the synthesis process leads to depletion of the non-dominant monoamine.

The basolateral monoamine transporter of the proximal convoluted renal tubule cells is an organic cation transporter (OCT). The basolateral monoamine transporters of the proximal convoluted renal tubule cells are a “dual gate lumen transporter”. At entrance to the transporter is a serotonin gate and a dopamine gate. These gates when not fully open impede access to the transporter lumen by the associated monoamine. Attributes of this “dual gate lumen transporter” are as follows.

The serotonin and dopamine gates can exist in one of two states partially closed or open. In the partially closed state access to the transporter lumen by the monoamine associated with the gate is impeded. When a gate is partially closed this corresponds with phase 1, as previously discussed.

The serotonin and dopamine gates are never both partially closed at the same time. Only one of the gates has been observed as partially closed at any given time. While it is theoretically possible for both the serotonin gate or dopamine gate to be both partially closed simultaneously only a serotonin gate or a dopamine gate or neither have been observed in phase 1 at any point in time. As amino acid precursor dosing is increased or decreased the total amount of serotonin and dopamine presenting at the entrance to the transporter increases or decreases respectively causing a partially closed gate to open or close respective in direct relation to the total amount of serotonin and dopamine presenting at the transporter entrance. As a gate opens or closes monoamine associated with that gate is transported through the transporter lumen causing a drop or increase in the urinary monoamine transported respectively by the apical transporter of the proximal convoluted renal tubule cells. While the term “gate” is used to illustrate the process of transporter observed with simultaneous manipulation of serotonin and/or dopamine it is possible that other effects such as allosteric effects or other processes are responsible for the gate phenomenon described herein. The exact mechanism may involved many senarios. For the purpose of this invention it is the measured outcomes of transport and not the mechanism that defines the teachings herein.

With increase in the total amount of monoamine presenting at the transporter, the point at which a gate is fully open is where phase 2, as previously discussed, starts. In phase 2 transport is not saturated causing the urinary monoamine levels to be below the therapeutic range.

As the total amount of monoamine presenting at the transporter increases, the point in phase 2 at which the transporter becomes saturated is where phase 3 starts. An increase in amino acid precursor dosing in phase 3 leads to increase in the associated the urinary monoamine due to transporter saturation. In phase 3 the transporter is saturated and in the competitive inhibition state.

When serotonin and/or dopamine are in the competitive inhibition state, functions traditionally attributed to only dopamine, or to only serotonin, may be regulated by manipulation of either monoamine. For example, sodium transport is recognized as being regulated by dopamine via transporter regulation. When both serotonin and dopamine are in the competitive inhibition state manipulation of either dopamine or serotonin will affect change to the amount of dopamine transported and the control of the process may be affected by manipulation of only serotonin. This interaction allows for understanding of how both serotonin and dopamine can control all functions attributed to only serotonin or dopamine, when serotonin and dopamine are in the competitive inhibition state.

Serotonin and dopamine function as one system in the competitive inhibition state and manipulation of either or both can be utilized to control function traditionally recognized as being controlled by only one.

The following are examples of functions directly or indirectly regulated by serotonin and/or dopamine. The list includes but is not limited to:
1. Regulation of phosphate.
2. Loss of serotonin transporters associated with Irritable Bowel Syndrome.
3. Hyperammonemia.
4. Hyperammonemia associated with retardation.
5. Regulation of alterations in diabetes.
6. Regulation of renal function.
7. Regulation of renal hemodynamics.
8. Blood pressure regulation.
9. Potassium regulation.
10. Sodium regulation.
11. ATP regulation.
12. Regulation of receptors outside the central nervous system, including as but not limited to:
   a. adrenal gland;
   b. blood vessels;
   c. carotid body;
   d. intestines;
   e. heart;
   f parathyroid gland;
   g. kidney; and
   h. Urinary tract.
13. Regulation of rennin secretion.
14. Regulation by autocrine or paracrine fashion.
15. Regulation in essential hypertension
16. Regulation of angiotensin II.
17. Regulatory functions in the evolution of shock.
18. Regulatory functions in the evolution of septic shock.
19. Regulation of oxidative stress.
20. Regulation of glomerular filtration.
21. Regulation of functions that strengthen, examples including but are not limited to:
   a. Bone marrow;
   b. Spleen, and
   c. Lymph nodes.
22. Regulation of dopamine in bone marrow cells to include but not limited to:
   a. Splenocytes; and
   b. Lymphocytes from lymph nodes.
23. Regulation in idiopathic hypertension.
24. Regulation of the sympathetic nervous system.
25. Regulation of platelet function.
26. Regulation in prostate cancer.
27. Regulation of syncope due to carotid sinus hypersensitivity.
28. Regulation of dialysis hypotension.
29. Regulation of cardiophysiologic function.
30. Regulation of adrenochromaffin cells.
31. Regulation in hypoxia induced pulmonary hypertension.
32. Regulation in Tourette's Syndrome.
33. Regulation of drug absorption and elimination.
34. Regulation in preeclampsia.
35. Regulation of modulation of fluid and sodium intake via actions to include but not limited to:
   a. in the central nervous system; and
   b. gastrointestinal tract
36. Regulation oftubular epithelial transport.
37. Regulates modulation of the secretion and/or action of vasopressin which in turn causes change in but not limited to:
   a. Rennin;
   b. Aldosterone;
   c. Norepinephrine;
   d. Epinephrine; and
   e. Endothelin B receptors (ETB) receptors.
38. Regulates fluid and sodium intake by way of “appetite” centers in the brain.
39. Regulates alterations of gastrointestinal tract transport.
40. Regulates detoxification of exogenous organic cations.
41. Regulation of prolactin secretion.
42. Regulation affecting memory.
43. Regulation of receptors in the central and peripheral system.
44. Regulation of fluid and electrolytes balance to include but no limited to;
   a. Blood vessels;
   b. Gastrointestinal tract;
   c. Adrenal glands;
   d. Sympathetic nervous system;
   e. Hypothalmus; and
   f Other brain centers.
45. Regulation of phosphorylation of DARPP-32.
46. Regulation of dependent effects of psychostimulants and opioids.
47. Regulation of neuronal differentiation.
48. Regulation of neurotoxicity.
49. Regulation of transcription.
50. Regulatory effects on fibroblasts.
51. Regulation of melatonin synthesis in photoreceptors.
52. Cyclic regulation of intraocular pressure.

The listing above is in not conclusive and is identified in order to give perspective on the wide range of functions regulated directly or indirectly by serotonin and/or dopamine.

This invention involves the administration of amino acid precursors of serotonin and dopamine with or without urinary serotonin and dopamine assay and interpretation of the phase as described above, in order to affect optimal regulation of function by serotonin and/or dopamine.

The organic cation transports of the kidney are identical and homologous transporters to the organic cation transporters of the liver, intestine, and brain. Presumably there are other organic cation transporters in other places which are also identical and homologous. The amino acid precursors cross the blood brain barrier then achieve steady state throughout the body thereby affecting all transporters in a similar manner throughout the body. Manipulation of serotonin and dopamine with amino acid precursors and then monitoring the response of the organic cation transporters in one place, such as the kidneys, gives insight into the functional status of organic cation transporters in other places of the body.

Determination of the phase of urinary monoamines is a determination of the functional status of the basolateral monoamine transporters (OCT). In affecting determination, the status of the serotonin and dopamine gates and lumen saturation status is identified. In order to gain optimal control of functions by both serotonin and dopamine both serotonin and dopamine must be in phase 3 (the competitive inhibition state), as identified above. Proper interpretation of the results is required for optimization of transporter function in administration of amino acid precursors. The amino acid precursors cross the blood brain barrier and with consistent dosing come to equilibrium. Once steady state has been achieved the amino acids are in equilibrium in the central and peripheral systems. In equilibrium the amino acid precursors have a uniform effect on the organic cation transporters associated with precursor uptake in all places of the system being taken up in a similar manner. Secondary to uptake by the organic cation transporters in the kidneys and system where ever the aromatic-L-amino acid decarboxylase (AAAD) is found, the amino acid precursors are synthesized to serotonin and dopamine. Once synthesized, serotonin and dopamine are excreted out of the cells responsible for synthesis. Excretion places the serotonin and dopamine in an environment where they regulate function at the transporter, around the transporter or in the system. Regulation of function is dependent on the amount of monoamines transported with is dependent the amount of monoamine synthesized and metabolized. In the kidney uptake of serotonin and dopamine precursors by the organic cation transporters leading to synthesis of dopamine, and synthesis of serotonin, leading transport out of the cells, correlates with these processes cells in other places where the organic cation transporters are found and perform similar function throughout the body. By manipulating the serotonin and dopamine synthesis with amino acid precursors, the effects of organic cation transporters that excreted serotonin and dopamine into the system in other places is manipulated affecting control of function traditionally recognized as being regulated by only one monoamine by both serotonin and dopamine.

Serotonin and dopamine leave the proximal convoluted renal tubule cells via the basolateral monoamine transporter to the interstitium or they are excreted into the urine via the apical transporter. An assay of urinary serotonin and dopamine with proper interpretation is a direct measurement of serotonin and dopamine not taken up by the organic cation transporters (OCT) of the proximal convoluted tubule cells.

In one embodiment, the method makes provisions for administration of serotonin and dopamine precursors with or without laboratory assay and phase determination in order to optimize functions regulated by serotonin and/or dopamine. The subject is simultaneously administered precursors of serotonin and dopamine. Once the steady state of the serotonin and dopamine is achieved an objective observation is made to determine if the desired response has been affected. If the desired response is not achieved the dosing of the amino acid precursors is adjusted to further to obtain the desired response. Repeated observations and adjustments may be made. If no objective observation is available or adjustment of amino acid dosing dose does not achieve the desired objective response in the desired period of time, a urine sample is collected as identified above which is then analyzed to determine the serotonin and dopamine levels and phase.

Examples exist where the serotonin and dopamine phase can be determined with only one test while in other examples two assays on differing amino acid precursor dosing are required. For example in a subject taking a higher dose of serotonin precursor with a lower dose of dopamine precursor and both urine levels are above phase 2 identifies that the dopamine is phase 1 and the serotonin is phase 3 since only gate is partially closed if at all. In some embodiments phase determination requiring two assays is implemented, where the subject is administered a different dosing of a precursor or precursors of serotonin and dopamine for each assay. The urinary serotonin and dopamine concentrations of the first sample are compared with the urinary serotonin and dopamine concentrations of the second sample to determine the phase of the serotonin and dopamine for each sample.

When the organic cation transporters are in competitive inhibition (phase 3) manipulation of one monoamine by changing its amino acid precursor dosing will lead to change in transport of the other monoamine along with associated change in function regulated by the other monoamine. For example, in the competitive inhibition state if only 5-HTP or L-dopa is administered leading to elevated levels of serotonin or dopamine respectively, this will lead to inhibition of transport of dopamine and serotonin in the competitive inhibition state respectively as evidence by increased urinary levels of the monoamine who's precursor was not administered. Regulation of function by serotonin and dopamine is primarily controlled by transport.

An example, including, but not limited to, what happens is when both dopamine and serotonin are in the competitive inhibition state is identified as follows: With changes in serotonin, changes in angiotensin levels are observed. Prior to this invention control of angiotensin levels was thought to be exclusively regulated by dopamine and not affected by changes in serotonin levels. When serotonin and dopamine are in the competitive inhibition state all functions in the system traditionally thought of as being regulated by only serotonin and only dopamine may be regulated by manipulation of either.

The exact amount of serotonin and dopamine or their precursors needed to achieve the competitive inhibition state of both is not a fixed point and varies greatly in a population. For example, if the desired end point to achieve phase 3 serotonin and dopamine, both within three standard deviations of the high end of the reference range reported by the lab then the following considerations may exist: For example, serotonin and dopamine are found to both be in the competitive inhibition state (phase 3) with serotonin levels one standard deviation above the high end of reference range reported by the lab and dopamine levels three standard deviations above the high end of the reference range reported by the lab. Circumstances exist where it is desirable to achieve both the serotonin and dopamine three standard deviations above the high end of the reference range reported by the lab. A single amino acid precursor 5-hydroxytryptophan is added to the amino acid precursor dosing. Upon examination of the steady state urine following the addition of 5-hydroxytryptophan it is found that the urinary serotonin is now phase 3 and three standard deviations above the reference range reported by the lab, and the phase 3 dopamine is now five standard deviations above the reference range reported by the lab. Increasing serotonin levels while serotonin and dopamine are in competitive inhibition excludes dopamine from the transporter causing more dopamine to end up in the final urine. While urinary dopamine levels have in fact risen the transport of dopamine has decreased. The next step will be to decrease administration of the dopamine precursor in balance with increasing the dosing of serotonin precursor (in order to keep the urinary serotonin level stable while the dopamine precursor is decreased in order to affect the stated goal.

In one embodiment, the goal is to place both the serotonin and dopamine into the competitive inhibition state (phase 3) without achieving excess levels of either in the urine. Excessively high levels of serotonin or dopamine in the urine indicate too much amino acid precursor is being administered and that the system is synthesizing too much serotonin or dopamine leading exclusion of the non-dominant monoamine by the dominant monoamine. By achieving serotonin and dopamine in competitive inhibition (phase 3), at desired levels of within 3 standard deviations above the high end of the reference range reported by the laboratory, optimal regulation and manipulation of serotonin and/or dopamine can be achieved through balanced competitive inhibition.

Exceptions to the stated goal do occur. Examples include but are not limited to instances where there is damage to system structures that interact with serotonin or dopamine. In such cases phase 3 levels of serotonin or dopamine may be higher than 3 standard deviations above the high end of the reference range reported by the laboratory. In this case the goal continues to be achieving both the serotonin and dopamine in phase 3, but the urinary levels of one or the other or both may need to be relatively higher than is normally optimal in other states.

Amino acid precursor dosing ranges needed to achieve steady state balance vary widely within the population and from population to population. Dosing is independent of weight. In adult humans the dosing of 5-hydroxytryptophan needed for optimal balance is found to be 1 mg to 3,000 mg per day. In humans the dosing of L-dopa ranges from 1 mg to 25 grams per day. The tyrosine dosing varies from 250 mg to 24 grams per day. The phenylalanine and n-acetyl-tyrosine daily dosing varies from 1 mg to 10 grams per day. In human children the dosing is approximately half the adult dose. In non-human subjects which possess a kidney, serotonin, and dopamine system dosing is started at the rate of 25 mg per day on the serotonin precursor and 250 mg per day on the dopamine precursor. The dosing is not as important as to realize that the dosing ranges needed for proper balance are huge and very greatly. Proper balance in one subject may be obtained with very low dose serotonin precursor and very high dose dopamine precursor. Whereas the next subject may require the exact opposite of optimal balance, i.e. very high dose serotonin precursor and very low dose amino acid precursor. An orderly starting point in humans is serotonin precursor with dopamine precursor to withing 85% of a 10:1 ratio. This is a starting point and the final dosing for optimal response may be markedly different.

Urinary serotonin and dopamine in competitive inhibition levels, when interpreted properly, represent an assay of the functional status of not only the basolateral monoamine transporter (OCT), but an assay of the function status of other organic cation transporters in the body that are “identical and homologous”. This gives the ability to gauge the effects of changing amino acid precursor dosing in all places through out the body where ever identical and homologous OCT transporters are found. Contrary to assertions in the literature the competitive inhibition state of serotonin and dopamine is not meaningless. The teaching of this invention is the first teching ever that asserts that the competitive inhibition state of monoamines is not a meaningless theorectical with no meaning.

In some embodiments, the subject is simultaneously administered a serotonin and/or dopamine precursor(s). Once steady state of serotonin and dopamine is achieved, an objective observation is made to determine if the desired response has been affected. If the desired response has not been achieved, the dosing of the amino acid precursors is adjusted until the desired response is obtained. Repeated observations and adjustments may be made. If no objective observation is available or adjustment of the amino acid dosing does not achieve the desired objective response, a urine sample is collected and analyzed to determine urinary serotonin and dopamine levels. The subject is then administered a different dosing of serotonin and/or dopamine precursor(s). Once a steady state of the second dosing of serotonin and/or dopamine precursors, as well as serotonin and dopamine is achieved, a second urine sample is collected and analyzed to determine the serotonin and dopamine levels. The urinary serotonin and dopamine assay of the first sample is compared with the urinary serotonin and dopamine assay of the second sample. The comparisons are used to determine the phase of serotonin and the phase of dopamine in each sample and the status of OCT competitive inhibition versus OCT control.

In one embodiment, the inventive method regulates the function of The System through amino acid precursor administration whereby serotonin or dopamine will not be depleted through unbalance synthesis or unbalanced metabolism leading to regulation of functions controlled by one monoamine in the endogenous state may be performed by manipulation of either monoamine in the competitive inhibition state.

In at least one embodiment, it is highly desirable to establish the competitive inhibition state of both serotonin and dopamine.

In at least one embodiment, it is desirable to assess the functional status of a transporter involved in transporting both serotonin and dopamine in a subject possessing a kidney, serotonin synthesis metabolism and transport systems, along with dopamine synthesis metabolism and transport systems, where the subject is administered a precursor of serotonin selected from the list containing tryptophan or 5-hydroxytryptophan along with a precursor of dopamine selected from a list containing phenylalanine, N-acetyl-tyrosine, tyrosine (to induce all synonyms of tyrosine), or L-dopa.

In at least one embodiment the subject is initially provided, 5-hydroxytryptophan in a dosing range is 1 mg to 3,000 mg per day.

In at least one embodiment the subject is initially provided, L-dopa in a dosing range is 1 mg to 25 grams per day.

In at least one embodiment the subject is initially provided tyrosine in a dosing range is 25 mg to 24 grams per day.

In at least one embodiment the subject is initially provided, phenylalanine and n-acetyl-tyrosine in a daily dosing range is 1 mg to 10 grams per day.

After administering the amino acid precursor of serotonin with the amino acid precursor of dopamine consistently on a daily basis for enough time to achieve steady state equilibrium of the amino acid precursors, urinary serotonin, and urinary dopamine a urine sample is obtained. In general steady state equilibrium is reached in 3 to 5 days following the initiation of the dosing identified herein. In order to facilitate all subjects in the population obtaining steady state equilibrium, obtaining a urine sample in 7 or more days after initiation or a change in amino acid precursor dosing is desirable.

The optimal time for obtaining the above-identified urine sample cited is 5 to 6 hours prior to onset of the sleep cycle for the subject matter although other collection times may be used.

The urine sample may then be analyzed for the concentration of serotonin and dopamine in the urine by standard laboratory assay methods, including but not limited to RIA, ELISA, and HPLC.

The concentration of serotonin and dopamine in the urine may be further analyzed to compensate for dilution of the urine (specific gravity).

In at least one embodiment, the urinary serotonin and dopamine results is reported in units of “micrograms of serotonin or dopamine per gram of creatinine”.

In at least one embodiment, after the above-identified analysis has been performed, the dosing of the amino acid precursors of serotonin and/or dopamine may be increased or decreased. It is noted that a dosing change of only one or both of the amino acid precursors may be elected. A second urinary assay as identified above may then be performed.

In at least one embodiment, the first and second urinary serotonin and dopamine assays may be compared then interpreted to find the phase of urinary serotonin and dopamine, with considerations as follows:
a. In phase determination the total amount of serotonin and dopamine precursors directly correlates with the total amount of serotonin and dopamine presenting at the transporter.
b. Under the dual gate-lumen model increasing or decreasing the total amount of amino acid precursors administered inversely decreases or increases the amount of serotonin or dopamine in the urine in phase 1 as the gate associated with the serotonin or dopamine closes of opens respectively. A gate that is partially closed is phase 1. In phase 1 the transporter is not saturated with the monoamine associated with the gate.
c. In phase 2 the gate is open, the transporter is not saturated and the majority of serotonin or dopamine presenting at the transporter is transported causing the urinary serotonin or dopamine to be low (<the therapeutic range).
d. In phase 3 the gate is open and transport is saturated increases or decreases in serotonin or dopamine will cause increases or decreases in urinary serotonin and dopamine.

| | | | Phase 3 |
|---|---|---|---|
| | | | Competitive |
| | Phase 1 | Phase 2 | Inhibition |
| Gate status | Partially closed | Open | Open |
| Transporter state | Not Saturated | Not Saturated | Saturated |

Functions previously controlled by only serotonin or only dopamine in the endogenous state may be controlled by manipulation of either serotonin or dopamine or both when both serotonin and dopamine are placed in the competitive inhibition state. In 1996 one the leading (if not the leading) monoamine kidney researchers in the world Soares-da-Silva described the “competitive inhibition state” when he went onto say, “It is probably meaningless” since working only with serotonin or only with dopamine, he could see no effective way to manage the competitive inhibition state, i.e the simultaneous three phase testing with interpretation of serotonin and dopamine did not exit. In the competitive inhibition state serotonin and dopamine are both transported and compete with each other. The competitive inhibition state can only be controlled when provisions are made to manipulate both serotonin and dopamine simultaneously.

In at least one embodiment, the process of adjusting the amino acid precursor dosing of serotonin and dopamine to place serotonin and dopamine in the competitive inhibition state involves repeating the steps identified above followed by repeated adjustments to the serotonin and/or dopamine amino acid precursor dosing. This continues until both the urinary serotonin and dopamine are in the phase 3 competitive inhibition state. In competitive inhibition state the transport of both serotonin and dopamine are saturated, and both are competing with the other for transport. When both serotonin and dopamine are in the competitive inhibition state in the transporter the following properties are in place.

An increase or decrease in amino acid dosing of one precursor of serotonin or dopamine causes increase or decrease in transport of the monoamine associated with the precursors administered and a decrease or increase in transport of the other monoamine as it is competitively excluded or facilitated in transport. The net effect on the urinary levels of serotonin and dopamine is both increase or decrease when both are in phase 3 (competitive inhibition). To insure optimal balance in transport it is desirable to establish both serotonin and dopamine in phase 3, within three reference range standard deviations above the high end of the reference range reported by the laboratory, as an initial starting point for phase 3 manipulation

In some embodiments the method objectively places both the serotonin and dopamine in the competitive inhibition state as evidenced by urinary serotonin and dopamine in the phase 3 competitive inhibition state allowing for functions previously recognized in the endogenous state as being controlled by only serotonin or only dopamine to controlled by manipulation of either.

Classes of functions associated with serotonin or dopamine include:
1. Regulatory;
2. Neurohormone;
3. Paracrine; and
4. Autocrine.

In the endogenous state, inside of each of these four classes are functions whose changes are recognized as associated with changes in only one monoamine, in only dopamine or only serotonin. A method has not been known to manipulate the functions of these four classes by either serotonin or dopamine individually, or both in combination.

In general serotonin or dopamine:
a. Affect regulatory function after transport into the system away from the transporter.
b. A neurohormone is any hormone produced by neurosecretory cells, usually in the brain. Neurohormonal activity is distinguished from that of classical monoamines as it can have effects on cells distant from the source of the hormone.
c. Serotonin and dopamine may act as autocrine agents that bind to autocrine receptors on the transporter, leading to changes in the cells of the transporter.
d. Serotonin and dopamine may act as paracrine agents where the target cell is near the transporter. Autocrine signaling occurs among the same cell, paracrine signaling affects other cells near the transporter. Two neurons would be an example of a paracrine signal.
e. The monoamine may perform more than one of these functions carrying out regulation of one function. For example, neurohormone paracrine regulation exists.

In at least one embodiment, the actual regulation of these four functions is dependent on only serotonin or only dopamine binding to receptors. The amount of monoamine binding to the receptor is directly dependent on the amount of monoamine transported. The amount of serotonin or dopamine binding at the receptors can be regulated in the competitive inhibition state by manipulation of serotonin or dopamine or both.

In at least one embodiment, the endogenous state, dopamine is a potent vasopressor acting peripherally to increase blood pressure. The dopamine drip is a standard treatment method for dealing with shock in the endogenous state. In the endogenous state manipulation of only dopamine levels affect blood pressure problems associated with long term hypotension.

Placing the serotonin and dopamine in the competitive inhibition state allows for manipulation of serotonin to manipulate blood pressure. Increasing or decreasing serotonin excludes of facilitates dopamine transport leading to decreased or increased dopamine binding at the receptors regulating blood pressure. Plus, in the competitive inhibition state administration of dopamine over time will not deplete serotonin in a serious problem with long term dopamine drips.

In at least one embodiment, in the endogenous state dopamine inhibits Na⁺-K⁺-ATPase activity in the kidney. While in the endogenous state this process is controlled by only dopamine, placing serotonin and dopamine both in the competitive inhibition state allows for serotonin manipulation to regulate the process, as transport of dopamine is facilitated or excluded from the transport and access to the receptors change.

In at least one embodiment, dopamine via cyclic AMP and sodium constitute an autocrine and paracrine system regulating phosphate in the endogenous state. By placing both serotonin and dopamine in the competitive inhibition state manipulation of serotonin will facilitate or exclude dopamine transport thereby affecting change to cyclic AMP, sodium and phosphate levels. Serotonin regulation of function that may not be carried out in the endogenous state.

In at least one embodiment, in the endogenous state, serotonin has also been shown to facilitate the pituitary response to discrete neuroendocrine reflexes, such as the suckling-induced release of prolactin. This response is dependent on transport of serotonin to facilitate the binding of serotonin at the receptors. By placing both the serotonin and dopamine in the competitive inhibition state, manipulation of dopamine through facilitating or excluding serotonin in transport may regulate receptor binding of serotonin.

In at least one embodiment, in the endogenous state, the neuroendocrine hormone serotonin is a modulator of cholangiocyte proliferation. This process is dependent on transport of serotonin leading to receptor binding. In the endogenous state only serotonin regulates this process. By placing serotonin and dopamine in the competitive inhibition state transport leads to receptor binding may be controlled by manipulating dopamine. The net result is that dopamine manipulation may be used to control cholangiocyte proliferation.

In at least one embodiment, it is important to not simply use the monoamine associated in the endogenous state to attempt to regulate the function or system to receive treatment. It is important to place serotonin and dopamine into the competitive inhibition state. It is important to place serotonin and dopamine into the competitive inhibition state because the primary differentiation between the endogenous state and the competitive inhibition state is what happens with the use of only one monoamine amino acid precursor in the endogenous state, versus the use of both serotonin and dopamine precursors in the competitive inhibition state. The full scope of the competitive inhibition state involves consideration for synthesis, metabolism and transport. Administering only one monoamine precursor will inhibit synthesis, enhance metabolism, and inhibit transport of the other monoamine precursor and monoamine. With administration of only one monoamine precursor, depletion of the other monoamine occurs. When depletion of the other monoamine is significant, enough functions regulated by the primary monoamine will no longer respond to treatment, no matter how high the levels of the monoamine are established. For optimal outcomes the serotonin and dopamine should be in the proper balance of the competitive inhibition state.

Depletion of the non-dominant monoamine may occur not only when only one amino acid precursor is administered, it may be present at the start, or it may also occur when amino acid precursors of both serotonin and dopamine are administered in an improperly balanced manner. For example, a small amount of one precursor is administered with a large amount of the other precursor leading to depletion of the monoamine precursor associated with the smaller amount administered.

In at least one embodiment the method described herein is highly effective for addressing use of amino acid precursors of serotonin and dopamine in order to prevent depletion of one or the other, or in circumstance where extremely low levels of serotonin and dopamine exist at the start. When serotonin and dopamine are both in the therapeutic range of the competitive inhibition state, proper balanced is achieved and depletion of one of the monoamines is not occurring due to unbalanced synthesis, metabolism, or transport. Defining the optimal therapeutic range for each disease state leads to variability in therapeutic range definition.

In at least one embodiment the method described herein teaches a method to regulate function through amino acid precursor administration whereby serotonin or dopamine will not be depleted and regulation of function by one monoamine in the endogenous state may be performed by manipulation of either monoamine in the competitive inhibition state.

In the past, without the testing as identified herein, it was extremely difficult to place serotonin and dopamine into the competitive inhibition state, because individual requirements for dosing of amino acid precursors vary from subject to subject on a large scale. One subject may have serotonin in phase 3 on 37.5 milligram of 5-HTP per day, while another subject might need 1,800 mg per day of 5-HTP for placement into phase 3.

The embodiments and examples described above are intended to be illustrative and not limiting. Additional embodiments are within the claims below. Although the present invention has been described with reference to specific embodiments and examples, workers skilled in the art will recognize that changes may be made in form and detail without departing from the spirit and scope of the invention. In addition, the terms including, comprising and having as used herein are intended to have broad non-limiting scope. References cited above are incorporated to the extent that they are not inconsistent with the explicit disclosure herein.

What is claimed is:

1. A method to assess and to regulate monoamine systems and synthesis, metabolism, and transport functions for monoamine systems in a subject through amino acid precursor administration comprising:
a. Providing said subject with an initial dosage of at least one of; an amino acid precursor of serotonin, an amino acid precursor of dopamine, and both an amino acid precursor of serotonin and dopamine, for at least three days;
b. Acquiring an initial urine sample from said subject at a low point of the diurnal monoamine variation;
c. Testing the initial urine sample to identify a concentration for serotonin, dopamine, and creatinine by laboratory assay;
d. Calculating a first ratio of serotonin and dopamine from said first urine sample against creatinine;
e. Administering an altered dosage of at least one of; an amino acid precursor of serotonin, an amino acid precursor of dopamine, and both an amino acid precursor of serotonin and an amino acid precursor of dopamine, to said subject on a daily basis for a period of time of at least three days;
f. Acquiring a second urine sample from said subject at a low point of the diurnal monoamine variation, said acquiring of said second urine sample being obtained at least three days after said administering of said altered dosage;
g. Testing said second urine sample to identify a second concentration for serotonin, dopamine, and creatinine by laboratory assay;
h. Calculating a second ratio of serotonin and dopamine from said second urine sample as against creatinine;
i. Comparing said second ratio to said first ratio to determine a status for at least one of; said concentration of serotonin, said concentration of dopamine, and said concentration of both serotonin and dopamine relative to a competitive inhibition state for at least one of; serotonin, dopamine, and both serotonin and dopamine, and formulating dosing changes relative to said altered dosage whereby said status of at least one of; serotonin and dopamine, may be adjusted toward a therapeutic dosing range for said amino acid precursor of serotonin and said amino acid precursor of dopamine associated with said competitive inhibition state, whereby said transport function for said monoamine system may be regulated by manipulation of dosing of at least one of; an amino acid precursor of serotonin, an amino acid precursor of dopamine, and both an amino acid precursor of serotonin and an amino acid precursor of dopamine;
j. Administering said dosing changes of at least one of; an amino acid precursor of serotonin, an amino acid precursor of dopamine, and both an amino acid precursor of serotonin and an amino acid precursor of dopamine, to said subject on a daily basis for a period of time of at least three days;
k. Acquiring a third urine sample from said subject at a low point of the diurnal monoamine variation, said acquiring of said third urine sample being obtained at least three days after said administering of said dosing changes;
l. Testing said third urine sample to identify a third concentration for serotonin, dopamine, and creatinine by laboratory assay;
m. Calculating a third ratio of serotonin and dopamine from said third urine sample against creatinine;
n. Comparing said third ratio to said second ratio to determine a revised status for at least one of; said concentration of serotonin, said concentration of dopamine, and said concentration of both serotonin and dopamine relative to the competitive inhibition state for at least one of; serotonin, dopamine, and both serotonin and dopamine, whereby said synthesis, metabolism and transport functions controlled by said monoamine system may be regulated by manipulation of at least one of; an amino acid precursor of serotonin, an amino acid precursor of dopamine, and both an amino acid precursor of serotonin and an amino acid precursor of dopamine, when both serotonin and dopamine are placed in said competitive inhibition state, for alleviation of symptoms suffered by said subject as a result of monoamine neurotransmitter or regulatory dysfunction associated with at least one of a catecholamine system and a serotonin system.

2. The method of claim 1, said amino acid precursor of serotonin comprising tryptophan.

3. The method of claim 1, said amino acid precursor of serotonin comprising 5-hydroxytryptophan.

4. The method of claim 1, said amino acid precursor of dopamine comprising phenylalanine.

5. The method of claim 1, said amino acid precursor of dopamine comprising N-acetyl-tyrosine.

6. The method of claim 1, said amino acid precursor of dopamine comprising tyrosine.

7. The method of claim 1, said amino acid precursor of dopamine comprising L-dopa.

8. The method of claim 3, said administering a dosage of at least one of an amino acid precursor of serotonin comprising a dosing range for said 5-hydroxytryptophan of 1 mg to 3,000 mg per day.

9. The method of claim 7, said administering a dosage of at least one of an amino acid precursor of dopamine comprising a dosing range for said L-dopa of 1 mg to 25 grams per day.

10. The method of claim 6, said administering a dosage of at least one of an amino acid precursor of dopamine comprising a dosing range for said tyrosine of 1 mg to 24 grams per day.

11. The method of claim 5, said administering a dosage of at least one of an amino acid precursor of dopamine comprising a dosing range for said n-acetyl-tyrosine of 1 mg to 10 grams per day.

12. The method of claim 1, said administering said altered dosage of at least one of an amino acid precursor of serotonin, an amino acid precursor of dopamine, and an amino acid precursor of both serotonin and amino acid precursor of dopamine, comprising at least one of an increase and decrease in said altered dosage of said at least one amino acid precursor of serotonin, an amino acid precursor of dopamine, and both an amino acid precursor of serotonin and an amino acid precursor of dopamine.

13. The method of claim 12, said administering said readjusted dosage of at least one of an amino acid precursor of serotonin, an amino acid precursor of dopamine, and an amino acid precursor of both serotonin and amino acid precursor of dopamine, comprising at least one of an increase and decrease in said readjusted dosage of said at least one amino acid precursor of serotonin, an amino acid precursor of dopamine, and both an amino acid precursor of serotonin and an amino acid precursor of dopamine.

14. The method of claim 1, wherein said competitive inhibition state is achieved when the transport of both serotonin and dopamine are saturated, and both serotonin and dopamine are competing with each other for transport.

15. The method of claim 1, said monoamine system functions selected from the group consisting of a regulatory system, a neurohormone system, a paracrine system and an autocrine system.
